(19) United States
(12) Patent Application Publication
(10) Pub. No.: US 2026/0176638 A1
(43) Pub. Date: Jun. 25, 2026

(54) ANTI-TUMOR EFFECT, PREPARATION AND USE OF SCHISTOSOMA JAPONICUM EGGS AND SECRETED AND EXCRETED PROTEINS THEREOF

(71) Applicant: NAVAL MEDICAL UNIVERSITY, Shanghai (CN)

(72) Inventors: Weiqing PAN, Shanghai (CN); Xiaobin FAN, Shanghai (CN); Nanhang LEI, Shanghai (CN); Weihua YAN, Shanghai (CN); Dongmei ZHANG, Shanghai (CN)

(21) Appl. No.: 18/993,871

(22) PCT Filed: Jul. 13, 2023
(86) PCT No.: PCT/CN2023/107297

§ 371 Date: Oct. 31, 2025

(30) Foreign Application Priority Data
Jul. 13, 2022 (CN) 202210828154.4

(51) Int. Cl. C12N 15/70; C12N 15/85

(52) U.S. Cl. CPC C12N 15/70; C12N 15/85; A61K 2300/00

(57) ABSTRACT

Disclosed are Schistosoma japonicum eggs and the ingredients of secretions and excretions thereof, comprising eggs, egg culture supernatant, and egg secreted proteins. (hereinafter referred to as “Schistosoma eggs and secretions and excretions thereof” for short). A Schistosoma infection can significantly inhibit metastatic tumors of organs such as lungs, and an anti-tumor effect is mediated by the “Schistosoma eggs and secretions and excretions thereof”, which have the effect of inhibiting various host tumors, comprising lung cancer, liver cancer, melanoma, and hematological tumors. Two egg secreted proteins having an anti-tumor effect are further identified. The “Schistosoma eggs and secretions and excretions thereof” exert an anti-tumor effect by activating natural immunity such as alveolar macrophages. The “Schistosoma eggs and secretions and excretions thereof” have a potential application value in prevention and treatment of tumors in humans.

### TECHNICAL FIELD

The present invention belongs to the fields of parasitology and oncology; More specifically, the present invention relates to the infection of Schistosoma japonicum and the isolation and preparation of its eggs, the preparation of the culture supernatant of its eggs, and the preparation of recombinant secreted and excreted proteins of the eggs using genetic engineering technology, and anti-tumor effects thereof.

### BACKGROUND

Schistosomiasis is a zoonotic parasitic disease caused by the parasitic infection of Schistosoma in humans or mammals. Among them, Schistosoma japonicum, Schistosoma haematobium, and Schistosoma mansoni have a wide range of prevalence and great harm, and only Schistosoma japonicum is prevalent in China. The eggs produced by adult Schistosoma japonicum can deposit in the liver. After the deposited eggs mature, the miracidia in the eggs secrete and excrete egg antigens (hereinafter referred to as “secreted and excreted proteins of eggs”), which penetrate into the surrounding liver tissue, causing aggregation of lymphocytes, macrophages, neutrophils, eosinophils, etc. around the eggs, forming egg granulomas, and subsequently leading to hepatic fibrotic lesions and resulting in liver cirrhosis.

In the early stage of host infection with Schistosoma japonicum, natural immune cells represented by liver macrophages play an important role in the initial inflammatory response. Surface molecules or secreted factors of Schistosoma activate macrophages, promoting M1-type macrophages to express interleukin-1 (IL-1), IL-12, iNOS, etc. At the same time, macrophages present Schistosoma antigens to T cells, further promoting the production of inflammatory cytokines and chemokines, thereby promoting the clearance of pathogens and causing inflammatory responses and host damage. In the late stage of infection, M2-type macrophages are activated and secrete pro-fibrotic cytokines such as IL-13 and Arginine-1 (Arg-1), which participate in inflammation resolution and tissue repair.

In recent years, the relationship between parasitic infections and the occurrence and development of tumors has gradually attracted attention and been researched. Although some parasites such as Clonorchis sinensis have been classified as biological carcinogens, reports from different laboratories have indicated a negative correlation between parasitic infections and tumors. Epidemiological studies have shown that the incidence rate of echinococcosis is negatively correlated with the incidence rate of some types of solid tumors, while animal model studies have shown that Toxoplasma gondii inhibits the development of melanoma and lung adenocarcinoma in mice. The anti-tumor mechanisms mediated by these parasitic infections remain unclear, but inducing the body to produce anti-tumor innate immunity is a potential mechanism among them.

In terms of Schistosoma japonicum infection, Schistosoma japonicum eggs are the main pathogenic factors of the disease. The deposition of a large number of eggs in the liver can cause chronic inflammatory granulomas in the liver, further leading to liver fibrosis and ultimately resulting in liver cirrhosis. Chronic inflammation and fibrosis are important risk factors for liver cancer, and liver cirrhosis itself is an independent carcinogenic factor. In patients with the same underlying diseases, the presence of liver cirrhosis can increase the incidence of liver cancer by 30-fold. In the 1960s and 1970s, in some severely endemic areas of schistosomiasis in China, over 70% of the population was infected with Schistosoma japonicum, and many patients progressed to the stage of liver cirrhosis. However, the association between Schistosoma japonicum infection and liver cancer has not been confirmed, and some studies suggest that there is no direct relationship between Schistosoma japonicum infection and malignant tumors in liver or intestine. Although there are currently a few studies reporting that Schistosoma japonicum infection may be associated with the occurrence of liver cancer, these studies are mostly retrospective clinical studies. In addition, there may also be the role of co-infection with hepatitis C virus and its contribution to hepatocarcinogenesis.

### SUMMARY OF THE INVENTION

The purpose of the present invention is to provide eggs produced by Schistosoma japonicum infection and secreted and excreted products thereof, as well as recombinant secreted and excreted proteins of the eggs, for use in the prevention and treatment of human tumors.

Another purpose of the present invention is, through extensive screening and identification of secreted and excreted proteins of eggs, to discover secreted and excreted proteins of Schistosoma japonicum eggs capable of inducing the host to produce anti-tumor effects, and to prepare recombinant proteins for the treatment and prevention of tumors.

In the first aspect of the present invention, it provides the use of a substance in the manufacture of a formulation or composition for (a) prevention and/or treatment of tumors; (b) activation of alveolar macrophages (AM); and/or (c) activation of innate immunity;
wherein, the substance is selected from the group consisting of:
(Z1) a schistosome egg polypeptide or a coding sequence thereof, or an expression vector expressing the egg polypeptide, wherein the egg polypeptide comprises: Sj-SP-19 or an active ingredient thereof, Sj-SP-489 or an active ingredient thereof, or a combination thereof;
(Z2) a fraction f4 of the live fertile egg culture supernatant (FES) of schistosome, wherein the fraction f4 is prepared using Millipore ultrafiltration centrifuge tubes with molecular weight cut-offs of 30 Kd and 50 Kd, and it is essentially composed of polypeptides with a molecular weight of approximately 30-50 Kd;
(Z3) a protein fraction from the live fertile egg culture supernatant (FES) of schistosome, wherein the protein fraction is free of or substantially free of components derived from schistosome, with the exception of proteins, and is free of or substantially free of components derived from any species, with the exception of Schistosoma;
(Z4) a live fertile egg culture supernatant (FES) of schistosome;
(Z5) any combination of Z1 to Z4 mentioned above.

In another preferred embodiment, the schistosome comprises Schistosoma japonicum and Schistosoma mansoni.

In another preferred embodiment, the egg polypeptide comprises wild-type and mutant forms of the egg polypeptide.

In another preferred embodiment, the egg polypeptide comprises an active fragment of the egg polypeptide.

In another preferred embodiment, the egg polypeptide comprises a pharmaceutically acceptable salt or ester of the egg polypeptide or an active fragment thereof.

In another preferred embodiment, the activation of alveolar macrophages refers to upregulating the expression of immune effector molecules such as IL-1β, TNF-α, and IL-12 and/or anti-tumor related factors and/or signaling pathways in the alveolar macrophages.

In another preferred embodiment, the innate immunity is the innate immunity in mammals.

In another preferred embodiment, the tumor comprises: lung cancer, liver cancer, melanoma, leukemia, malignant lymphoma, renal cancer, oral epithelial cancer, head and neck cancer, brain tumor, glioma, gastric cancer, esophageal cancer, ovarian cancer, colorectal cancer, cervical cancer, pancreatic cancer, prostate cancer, or breast cancer.

In another preferred embodiment, the tumor is selected from the group consisting of: lung cancer, liver cancer, melanoma, leukemia, malignant lymphoma, breast cancer, brain cancer, prostate cancer, ovarian cancer, cervical cancer, colorectal cancer, osteosarcoma, and pancreatic cancer.

In another preferred embodiment, the tumor is selected from the group consisting of: lung cancer, liver cancer, melanoma, leukemia, and malignant lymphoma.

In another preferred embodiment, the formulation or composition is used to inhibit the formation of metastatic tumors.

In another preferred embodiment, the metastatic tumor comprises a lung metastatic tumor, a liver metastatic tumor, a bone metastatic tumor, a brain metastatic tumor, or a combination thereof.

In another preferred embodiment, the metastasis comprises a lung metastasis from liver cancer, a lung metastasis from breast cancer, a lung metastasis from melanoma, a lung metastasis from gastric cancer and colorectal cancer, a brain metastasis from lung cancer, a bone metastasis from lung cancer, or a combination thereof.

In another preferred embodiment, the schistosome egg polypeptide comprises recombinant, artificially synthesized, or natural Sj-SP-19 and Sj-SP-489 polypeptides.

In another preferred embodiment, the amino acid sequence of the schistosome egg polypeptide Sj-SP-19 is as set forth in SEQ ID NO: 1.

In another preferred embodiment, the amino acid sequence of the schistosome egg polypeptide Sj-SP-489 is as set forth in SEQ ID NO: 2.

In another preferred embodiment, the egg polypeptide comprises a recombinant polypeptide with a tag sequence.

In another preferred embodiment, the recombinant polypeptide with a tag sequence is as set forth in SEQ ID NO: 3 or SEQ ID NO: 4.

In another preferred embodiment, the amino acid sequence of SjHis-SP-19, which is Sj-SP-19 with a His tag, is as set forth in SEQ ID NO: 3.

In another preferred embodiment, the amino acid sequence of SjHis-SP-489, which is Sj-SP-489 with a His tag, is as set forth in SEQ ID NO: 4.

In another preferred embodiment, the schistosome egg polypeptide comprises an amino acid sequence obtained by substitution, deletion, or insertion of one or more amino acids in the sequence of SEQ ID NO: 1 or SEQ ID NO: 2, within the scope of maintaining its polypeptide activity.

In another preferred embodiment, the schistosome egg polypeptide comprises an amino acid sequence obtained by insertion of one or more amino acids at the N-terminus or C-terminus of the sequence of SEQ ID NO: 1 or SEQ ID NO: 2, within the scope of maintaining its polypeptide activity; the number of inserted amino acid residues comprises 1-35, preferably 1-15, and more preferably 1-10.

In another preferred embodiment, the schistosome egg polypeptide comprises a recombinant protein with one or more protein tags at the N-terminus or C-terminus of the sequence of SEQ ID NO: 1 or SEQ ID NO: 2, within the scope of maintaining its polypeptide activity.

In another preferred embodiment, the protein tag is selected from the group consisting of: MBP tag, His tag, GST tag, SUMO tag, TRX tag, HA tag, Flag tag, and a combination thereof.

In another preferred embodiment, the coding sequence of the schistosome egg polypeptide is as set forth in SEQ ID NO: 5 or SEQ ID NO: 6.

In the second aspect of the present invention, it provides a pharmaceutical composition, comprising (a) a pharmaceutically acceptable carrier and (b) an active ingredient, wherein the active ingredient is selected from the group consisting of:
(Z1) a schistosome egg polypeptide or a coding sequence thereof, or an expression vector expressing the egg polypeptide, wherein the egg polypeptide comprises: Sj-SP-19, Sj-SP-489, or a combination thereof;
(Z2) a fraction f4 of the live fertile egg culture supernatant (FES) of schistosome, wherein the fraction f4 is prepared using Millipore ultrafiltration centrifuge tubes with molecular weight cut-offs of 30 Kd and 50 Kd, and it is essentially composed of polypeptides with a molecular weight of approximately 30-50 Kd;
(Z3) a protein fraction from the live fertile egg culture supernatant (FES) of schistosome, wherein the protein fraction is free of or substantially free of components derived from schistosome, with the exception of proteins, and is free of or substantially free of components derived from any species, with the exception of Schistosoma;
(Z4) a live fertile egg culture supernatant (FES) of schistosome;
(Z5) any combination of Z1 to Z4 mentioned above.

In another preferred embodiment, the component (b) accounts for 0.1-99.9 wt %, preferably 10-99.9 wt %, and more preferably 70%-99.9 wt % of the total weight of the pharmaceutical composition.

In another preferred embodiment, the formulation or combination can be used alone or in combination.

In another preferred embodiment, the pharmaceutical composition further comprises: (c) a second active ingredient, wherein the second active ingredient is an additional anti-tumor therapeutic medicament selected from the group consisting of: a chemotherapeutic medicament, an antibody medicament, and a combination thereof.

In another preferred embodiment, the dosage form of the pharmaceutical composition is a liquid dosage form.

In another preferred embodiment, the dosage form of the pharmaceutical composition is a liposomal formulation.

In another preferred embodiment, the pharmaceutical composition is a liquid, solid, or semi-solid composition.

In another preferred embodiment, the pharmaceutical composition is a liquid composition.

In another preferred embodiment, the dosage form of the pharmaceutical composition is an injection or a topical dosage form.

In another preferred embodiment, the dosage form of the pharmaceutical composition comprises an injection or a lyophilized formulation.

In another preferred embodiment, the dosage form of the pharmaceutical composition is an injection.

In another preferred embodiment, the pharmaceutically acceptable carrier is selected from the group consisting of: infusion carriers and/or injection carriers; preferably, the carrier is one or more carriers selected from the group consisting of: physiological saline, glucose saline, and a combination thereof.

In another preferred embodiment, the first active ingredient is a polypeptide having the core sequence as set forth in SEQ ID NO: 1 and/or SEQ ID NO: 2, or a mutant thereof within the scope of maintaining its polypeptide activity.

In another preferred embodiment, the first active ingredient is an expression vector that expresses a polypeptide having the core sequence as set forth in SEQ ID NO: 1 and/or SEQ ID NO: 2, or a mutant thereof within the scope of maintaining its polypeptide activity.

In another preferred embodiment, the expression vector comprises a plasmid.

In another preferred embodiment, the expression vector or plasmid comprises a promoter, a replication origin, and a marker gene.

In another preferred embodiment, the expression vector comprises an expression cassette for expressing the polypeptide.

In another preferred embodiment, the administration method of the pharmaceutical composition comprises: respiratory administration, injection administration, transdermal administration, and mucosal administration.

In another preferred embodiment, the pharmaceutical composition is administered in a manner selected from the group consisting of: subcutaneous injection, intramuscular injection, and intravenous injection.

In another preferred embodiment, the dosage form of the pharmaceutical composition comprises a spray, an aerosol, a powder aerosol or a suppository.

In another preferred embodiment, the subject comprises mammals.

In another preferred embodiment, the mammals comprise human or non-human mammals.

In another preferred embodiment, the non-human mammals comprise: rodents (such as rats and mice) and primates (such as monkeys).

In the third aspect of the present invention, it provides an effective fraction, which is a fraction f4 of FES (approximately 30-50 Kd).

In the fourth aspect of the present invention, it provides an egg polypeptide combination, which is essentially composed of Sj-SP-19 and Sj-SP-489, or a fusion protein of Sj-SP-19 and Sj-SP-489.

In another preferred embodiment, the total content of SjHis-SP-19 and SjHis-SP-489, or the content of the fusion protein of Sj-SP-19 and Sj-SP-489, in the egg polypeptide combination, is ≥90 wt %, preferably ≥95 wt %, and more preferably ≥99 wt %, calculated by the total weight of all polypeptides in the egg polypeptide combination.

In the fifth aspect of the present invention, it provides a nucleic acid combination, which is essentially composed of a first nucleic acid encoding Sj-SP-19 and a second nucleic acid encoding Sj-SP-489.

In another preferred embodiment, the first nucleic acid and the second nucleic acid are each independently linear or located on an expression vector.

In another preferred embodiment, the first nucleic acid is as set forth in SEQ ID NO: 5.

In another preferred embodiment, the second nucleic acid is as set forth in SEQ ID NO: 6.

In the sixth aspect of the present invention, it provides a use of the effective fraction according to the third aspect of the present invention, the egg polypeptide combination according to the fourth aspect of the present invention, the nucleic acid combination according to the fifth aspect of the present invention, or the pharmaceutical composition according to the second aspect of the present invention, in the manufacture of a medicament for (a) prevention and/or treatment of tumors; (b) activation of alveolar macrophages (AM); and/or (c) activation of innate immunity.

In the seventh aspect of the present invention, it provides a method for in vitro activation of alveolar macrophages, wherein alveolar macrophages are cultured in the presence of a substance to obtain activated alveolar macrophages, and the substance is described in the first aspect of the present invention.

In another preferred embodiment, the concentration of the substance used for activating alveolar macrophages is greater than 10 μg˜1000 μg/mL, and the culture time is 24˜72 hours.

In the eighth aspect of the present invention, it provides an activated alveolar macrophage prepared using the method of claim 7.

In another preferred embodiment, the activated AM cells have one or more of the following characteristics:
(Y1) up-regulated IL-1β expression;
(Y2) up-regulated TNF-α expression;
(Y3) up-regulated IL-12 expression;
(Y4) up-regulated Nos2 expression.

In the ninth aspect of the present invention, it provides a cell formulation or a pharmaceutical composition, which comprises the activated alveolar macrophage according to the eight aspect of the present invention and a pharmaceutically acceptable carrier.

In the tenth aspect of the present invention, it provides a use of the activated alveolar macrophage according to the eight aspect of the present invention, in the manufacture of a medicament for prevention and/or treatment of tumors.

In another preferred embodiment, the use comprises intravenous infusion of the activated alveolar macrophage to a subject in need thereof, thereby achieving the purpose of preventing and/or treating tumors.

In the eleventh aspect of the present invention, it provides a method for treating tumors, comprising a step of: administering a safe and effective amount of an active ingredient or a pharmaceutical composition comprising the active ingredient to a subject in need thereof, thereby treating the tumors of the subject, wherein the active ingredient is selected from the group consisting of: a substance as described in the first aspect of the present invention; the activated alveolar macrophage according to the eighth aspect of the present invention; and a combination thereof.

In another preferred embodiment, when the active ingredient is a substance as described in the first aspect of the present invention, the dosage administered is 0.05-10 mg/kg, preferably 0.1-5 mg/kg.

In another preferred embodiment, when the active ingredient is the activated alveolar macrophage of claim 8, the dosage administered is 5×10⁵ cells per mouse or 10⁶-10¹⁰ cells/60 kg per human.

It should be understood that within the scope of the present invention, each technical features of the present invention described above and in the following (such as examples) may be combined with each other to form a new or preferred technical solution, which is not redundantly repeated one by one herein due to space limitation.

### DESCRIPTION OF DRAWINGS

FIG. 1 shows the inhibitory effect of Schistosoma japonicum infection on lung metastatic tumors. Panel A, Experimental design schematic diagram; Panel B, LLC mouse model for lung adenocarcinoma; Panel C, B16 melanoma mouse model.

FIG. 2 shows that Schistosoma japonicum eggs inhibit lung metastatic tumors in mice. Panel A, Experimental design; Panel B, Live fertile eggs inhibit the metastasis of LLC tumor cells in the lungs; Panel C, Live fertile eggs prolong the survival period of mice; Live fertile eggs (F-egg); Boiled-inactivated dead eggs (D-egg); Panel D, Freshly isolated Schistosoma japonicum eggs.

FIG. 3 shows that live fertile eggs of Schistosoma japonicum inhibit lung metastatic tumors in NOD-SCID mice. Panel A, LLC tumor cell model; Panel B, B16 tumor cell model; F-egg: Live fertile eggs; D-egg: Boiled-inactivated dead eggs.

FIG. 4 shows the inhibitory effect of live fertile eggs deposited in the lungs on liver metastatic tumors. The experiment was conducted using NOD-SCID mice and a B16 tumor cell model; F-egg: Live fertile eggs; D-egg: Boiled-inactivated dead eggs.

FIG. 5 shows the inhibitory effect of egg culture supernatants on lung and liver tumors. FES: Live fertile egg culture supernatant; DES: Dead egg culture supernatant; Control: Culture medium control.

FIG. 6 shows the dynamic analysis of immune cell changes in the lungs induced by eggs. Panel A, Dynamic analysis of changes in the number of T cells, B cells, and NK cells in the bronchoalveolar lavage fluid (upper) and lung tissue (lower) of mice after injection of eggs or PBS by flow cytometry. Panel B, Panel C, Dynamic analysis of changes in the number of alveolar macrophages (AMs) (Panel B) and other types of macrophages (CD11c-) (Panel C) in mouse lung tissue after injection of eggs or PBS. Two-way analysis of variance and Sidak post-hoc test were used. * P<0.05, **P<0.01, ***P<0.001.

FIG. 7 shows the anti-tumor effect mediated by alveolar macrophages. Panel A, Mice were injected with live fertile eggs and administered with clodronate liposomes via bronchus, and the proportion of AMs in bronchoalveolar lavage fluid was detected by flow cytometry. Panel B, The changes in the number of lung metastatic tumors after injection of live fertile eggs and clearance of AMs in C57BL/6 mice (LLC tumor model) or NOD-SCID mice (B16-F10 tumor model). Panel C, The proportion of AMs in bronchoalveolar lavage fluid of mice administered with clodronate liposomes via bronchus and infected with Schistosoma japonicum. Panel D, The changes in the number of lung metastatic tumors in C57BL/6 mice (B16-F10 tumor model) infected with Schistosoma japonicum. T-test or one-way analysis of variance was used. ** P<0.01, ***P<0.001.

FIG. 8 shows the in vitro killing effect of FES-activated AMs on tumor cells. Panel A, Primary AMs were isolated from mice injected with eggs or PBS, and their killing effect on LLC tumor cells was detected by high-content assay. Scale bar=125 μm. Panel B, MH-S cells stimulated with different concentrations of FES were co-cultured with B16-GFP/Luc, and the proportion of GFP and F4/80 double positive MH-S cells (i.e., MH-S cells phagocytosing B16-GFP/Luc) among the total MH-S cells was detected by flow cytometry. Panel C, Primary AMs were isolated from mice injected with FES or PBS and co-cultured with B16-GFp/Luc at a ratio of 4:1. The proportion of GFP and F4/80 double positive AMs (i.e., AMs phagocytosing B16-GFP/Luc) among the total AMs was detected by flow cytometry. Panel D, Primary AMs were isolated from mice injected with eggs or PBS and transfused back into C57BL/6 mice (LLC tumor model) or NOD-SCID mice (B16-F10 tumor model). The number of lung metastatic tumors was then counted. T-test or one-way analysis of variance and Tukey post-hoc test were used. * P<0.05, **P<0.01, ***P<0.001.

FIG. 9 shows single-cell sequencing and clustering of three groups of AMs. Panel A, Schematic diagram of the workflow for single-cell sequencing (top) and flow cytometry sorting (bottom) of AMs. Panel B, Annotation information on cell types in the tSNE clustering results of 29052 original cells. Panel C, Panel D, Cell clustering information (Panel C) and sample distribution information (Panel D) in the tSNE clustering results of 27796 AMs. Panel E, The distribution of sample sources for each subpopulation of cells.

FIG. 10 shows the polarization phenotype of AMs and enrichment analysis of anti-tumor functional gene sets. Panel A, The average expression levels of M1 macrophage marker genes in different samples. Panel B, Violin plots of expression levels of partial M1 marker genes. Panels C-E, Enrichment analysis of gene sets related to oxidative stress, inflammasome, and phagocytosis. The empirical cumulative distribution plots (left) and related gene expression levels (right) of enrichment scores for oxidative stress (Panel C), inflammasome (Panel D), and phagocytosis (Panel E) in different samples.

FIG. 11 shows the identification of anti-tumor effector molecules in FES-activated AMs. Panel A, Comparison of the enrichment scores for cell killing related gene sets in different samples. Panel B, Heatmap of expression differences in cytokines related to tumor suppression across different samples. Panel C, Panel D, Expression level dot plots (Panel C) and tSNE clustering plots (Panel D) for partial cytokines in Panel B.

FIG. 12 shows the experimental analysis of anti-tumor effector molecule IL-1β in FES-activated AMs. Panel A, The expression level of Il1b mRNA in AMs of mice injected with eggs or PBS. Panel B, The expression level of mature IL-1β in the serum of mice injected with eggs or PBS. Panel C, The expression level of mature IL-1β in the serum of mice injected with FES or PBS. Panel D, The number of lung metastatic tumors in C57BL/6 mice (B16-F10 tumor model) after injection of FES and IL-1β antibody (B122). Panel E, The number of lung metastatic tumors in IL-1β^{-/-} mice after injection of FES. T-test or one-way analysis of variance was used. * P<0.05, **P<0.01, ***P<0.001.

FIG. 13 shows the effect of treating FES with different enzyme on MH-S activation. FES treated with Proteinase K, DNase I, and RNase A were added to MH-S cells and cultured for 24 hours. RT-qPCR was performed to measure the mRNA expression levels of Il1b, Marco, and Nos2 in the cells. The data represent the relative expression levels of mRNA in each group relative to the Medium Control group, with GAPDH as the internal reference gene, and are presented as Mean±SD, N=4. Multivariate analysis of variance was used for data analysis, ***, P<0.001.

FIG. 14 shows the activation of MH-S cells by FES fractions and their anti-tumor effects in vivo. Panel A, Using Millipore ultrafiltration centrifuge tubes with different molecular weight cutoffs, FES was prepared into five fractions with different molecular weight ranges (f1 to f5), i.e., f1<3 kDa; 3 kDa<f2<10 kDa; 10 kDa<f3<30 kDa; 30 kDa<f4<50 kDa and f5>50 kDa. FES fractions containing proteins of different molecular weights were added to MH-S cells and the cells were cultured for 24 hours, after which RT-qPCR was performed to measure the mRNA expression levels of Il1b, Marco, and Nos2 in the cells. The data represent the relative expression levels of mRNA in each group relative to the Medium group, with GAPDH as the internal reference gene, and are presented as Mean±SD, N=4, ***, P<0.001; Panel B, Panel C, In vivo B16/F10 cell mouse model, B, Experimental design schematic diagram; C, The number of metastatic tumors in the lungs and liver of mice; Panel D, Representative HE-stained pathological sections of metastatic tumors in mouse lung tissues. The number of liver and lung tumors in mice, presented as Mean±SD, N=6, **, P<0.01. Medium: Culture medium control; FES: Egg culture supernatant; f4: FES containing proteins with molecular weights of 30-50 kDa.

FIG. 15 shows the screening of effector proteins for IL-1β expression in MH-S activated by f4 fraction recombinant protein. Panel A, Electrophoresis image of induced expression of partial proteins. IB: The precipitation portion of Escherichia coli lysate after centrifugation; SS: The supernatant portion of Escherichia coli lysate after centrifugation; Panel B, Protein electrophoresis image of purified partial proteins; Panel C, The expression level of Il1b mRNA in MH-S cells (alveolar macrophage cell line) stimulated by the recombinant protein. The numerical values are presented as Mean±SD, N=3, **, P<0.01, and the statistical analysis was conducted using one-way analysis of variance. Panel D, The effect of protease K-treated recombinant protein on the expression of Il1b mRNA in MH-S cells.

FIG. 16 shows the screening of effector proteins for IL-1β expression in MH-S activated by SjGST-SP recombinant protein. Panel A, SDS-PAGE electrophoresis image of partial expression products of SjGST-SP protein; Panel B, Detection of IL-1β secretion in MH-S stimulated with 183 kinds of SjGST-SP recombinant proteins; Panel C, Results of repeated experiments for four proteins with positive initial screening results. The data are presented as the ratio of the light intensity of the sample to that of the negative control, R=(average of two replicates of the sample−average of two replicates of the blank)/(average of two replicates of the negative control−average of two replicates of the blank); Panel D, SDS-PAGE electrophoresis images of SjHis-SP-57 and SjHis-SP-489 recombinant proteins, IB: The precipitate of Escherichia coli lysate after centrifugation; SS: The supernatant of Escherichia coli lysate after centrifugation; Panel E, Detection of the level of IL-1β secretion in MH-S upon stimulation, wherein Ctr1 and Ctr2 groups are irrelevant recombinant proteins with His tags; Panel F, Western blot and protein mass spectrometry were used to analyze the SjSP-489 recombinant protein, and mouse anti-SjHis-SP-489 serum was used as the primary antibody to detect the SjSP-489 protein in FES (left). The amino acid sequences of two purified SjSP-489 recombinant proteins were analyzed by mass spectrometry. The red color indicates the part where the mass spectrometry analysis results match the amino acid sequence of the target protein.

FIG. 17 shows the inhibitory effect of effector proteins on tumor growth in mice. Panel A, Experimental protocol, with 8 mice per group and the antigen injection dose of 30 μg per administration; Panel B, The number of lung tumors in each group of mice; Panel C, The number of liver tumors in each group of mice; Panel D, Panel E, 8 mice per group, with antigen injection dose of 60 μg per administration. Number of lung tumors in each group of mice (Panel D); Number of liver tumors in each group of mice (Panel E). ***: Compared with the PBS group, P<0.01; NS: No significant difference compared to the PBS group. SjHis-SP-12 and 30 μg SjHis-SP-24 were negative proteins screened in vitro.

FIG. 18 shows the effect of effector proteins on activating mouse alveolar macrophages in vitro and in vivo. Panel A, Il1b; Panel B. The expression of 1112 mRNA. Data represent the relative expression levels of Il1b and 1112 mRNA in macrophages, with GAPDH as the internal reference and the PBS group as the control (n=3); Panel C, The percentage of IL-1β molecule-positive mouse alveolar macrophages in total macrophages; Panel D, The percentage of IL-12 molecule-positive mouse alveolar macrophages in total macrophages; Panel E, The concentration of IL-1β in mouse serum. ***: Extremely significant difference compared to the PBS group.

FIG. 19 shows the homologous sequence alignment of Sj-SP-19 (FN316857). Sj: Schistosoma japonicum; Sh: Schistosoma haematobium; Sm: Schistosoma mansoni; Ms: Mus musculus; Hs: Homo species.

FIG. 20 shows the homologous sequence alignment of Sj-SP-489 (AY814009). Sj: Schistosoma japonicum; Sh: Schistosoma haematobium; Sm: Schistosoma mansoni.

### DETAILED DESCRIPTION

After extensive and in-depth research, the inventors have unexpectedly discovered for the first time that the eggs of Schistosoma japonicum, along with their culture supernatant and secreted and excreted proteins, have anti-tumor effects such as activating alveolar macrophages and inhibiting tumor formation and metastasis.

Specifically, based on a phenomenon or pattern that a large number of patients previously infected with Schistosoma japonicum would develop liver fibrosis and even cirrhosis, but rarely progress from liver fibrosis and cirrhosis to liver cancer, the inventors thus proposes a hypothesis: Schistosoma japonicum infection can induce the host to generate anti-tumor immunity and enhance the host's resistance to tumor occurrence and development. If the schistosome-derived molecules that induce anti-tumor immunity can be further clarified through experiments, they can be transformed into a new method for preventing and treating such diseases. To this end, the present invention applied a Schistosoma japonicum-naturally infected mouse model and an egg granuloma lung model constructed with isolated and purified live eggs, and combined with mouse lung adenocarcinoma cell line (LLC) and melanoma cell line (B16) metastatic tumor models, to experimentally investigate this hypothesis. The results showed that natural Schistosoma japonicum infection, isolated and purified live eggs, and their culture supernatant can induce a strong anti-tumor effect in the host, and this anti-tumor effect is exerted through the activation of alveolar macrophages and their secretion of IL-1β, etc. Additionally, two secreted and excreted proteins of eggs were screened and identified as the schistosome-derived effector molecules for activating natural immunity and anti-tumor effects.

### Term

In order to make this disclosure easier to understand, certain technical and scientific terms are specifically defined below. Unless otherwise explicitly defined herein, all other technical and scientific terms used herein shall have the meanings commonly understood by those skilled in the art to which the present invention belongs. Before describing the present invention, it should be understood that the present invention is not limited to the specific methods and experimental conditions described, as such methods and conditions may vary. It should also be understood that the terms used herein are only intended to describe specific embodiments and are not intended to be limiting. The scope of the present invention shall be limited only by the appended claims.

Unless otherwise defined, all technical and scientific terms used herein shall have the same meanings as those commonly understood by those skilled in the art to which the present invention belongs. As used herein, when used in reference to specific enumerated values, the term “about” means that the value may vary by no more than 1% from the enumerated value. For example, as used herein, the expression “about 100” includes all values between 99 and 101 (e.g., 99.1, 99.2, 99.3, 99.4, etc.).

As used herein, the term “optional” or “optionally” means that the events or situations described subsequently can occur but are not required to occur, can exist but are not required to exist, and can be one, two, or three in number.

### Schistosome Egg Polypeptide

As used herein, the terms “schistosome egg polypeptide”, “protein of the present invention”, and “polypeptide of the present invention” can be used interchangeably, referring to proteins consisting of Sj-SP-19 (SEQ ID NO: 1) and/or Sj-SP-489 (SEQ ID NO: 2), and having the activity of activating alveolar macrophages and/or anti-cancer activity. It should be understood that this term includes not only the wild-type proteins of Sj-SP-19 (SEQ ID NO: 1) and/or Sj-SP-489 (SEQ ID NO: 2), but also includes SjHis-SP-19 and SjHis-SP-489 proteins with a His tag inserted at their N-terminus, as well as these proteins with other tag proteins inserted; it also includes mutant proteins thereof, as long as these mutations do not affect or substantially do not affect their activity and efficacy.

The amino acid sequences involved in the present invention are as follows:

Wild type Sj-SP-19 (SEQ ID NO: 1):
MVYMIKYDSTHGKFQGDVSVENGKLNVNGRLISVYCERDPLNIPWNKDG
AEYVVESTGVFTTIDKAQAHIKNDRAKKVIISAPSADAPMFVVGVNEKT
YDKSMSVVSNASCTTNCLAPLAKVINDNFEIVEGLMTTVHSFTATQKTV
DGPSSKLWRDGRGAFQNIIPASTGAAKAVGKVIPALNGKLTGMAFRVPT
ANVSVVDLTCRLGKGATYDQIKAVIKAAANGPLKGILEYTEDEVVSSDF
IGCTSSSIFDAKAGISLNNNFVKLVSWYDNEFGYSCRVVDLITHMHRVD
HS
Wild type Sj-SP-489 (SEQ ID NO: 2):
MNQIKPRILFLLVLLIDLYDRILASNYDQYIDRLTNDGKLLYDDYIKQN
PSLESALERLYTLQHPIFQEDYPGNYEITDKQWNAFLNEIDQAKLGRLQ
NNDADKPEMTYSNLDRKSNYELYPNTNNNNDKVLNEPSLTERRNEIAYQ
NPLWGEHKVTGGSSETGQWIDYALLGAGAQDLLDNESSVDNFNLSNEIE
SSHIESKVDNLPAYCDPPNPCPLNYKSHDLPSPCDHGIEDTIEFNRNWI
IRKMENGECSCDNEHMDSCPIESNENGDKNNFVSAQKADRKPYWVNPYL
RGESRKRLVAKKRVKRSHTSFPSFQVYHYNPYLMGSVHKTAVKKIGPYK
PSHEKYM
SjHis-SP-19 sequence with a His tag (SEQ ID NO: 3)
MGSSHHHHHHSSGLVPRGSHMASMTGGQQMGRGSMVYMIKYDSTHGKFQ
GDVSVENGKLNVNGRLISVYCERDPLNIPWNKDGAEYVVESTGVFTTID
KAQAHIKNDRAKKVIISAPSADAPMFVVGVNEKTYDKSMSVVSNASCTT
NCLAPLAKVINDNFEIVEGLMTTVHSFTATQKTVDGPSSKLWRDGRGAF
QNIIPASTGAAKAVGKVIPALNGKLTGMAFRVPTANVSVVDLTCRLGKG
ATYDQIKAVIKAAANGPLKGILEYTEDEVVSSDFIGCTSSSIFDAKAGI
SLNNNFVKLVSWYDNEFGYSCRVVDLITHMHRVDHS
SjHis-SP-489 sequence with a His tag (SEQ ID
NO: 4)
MGSSHHHHHHSSGLVPRGSHMASMTGGQQMGRGSMNQIKPRILFLLVLL
IDLYDRILASNYDQYIDRLTNDGKLLYDDYIKQNPSLESALERLYTLQH
PIFQEDYPGNYEITDKQWNAFLNEIDQAKLGRLQNNDADKPEMTYSNLD
RKSNYELYPNTNNNNDKVLNEPSLTERRNEIAYQNPLWGEHKVTGGSSE
TGQWIDYALLGAGAQDLLDNESSVDNENLSNEIESSHIESKVDNLPAYC
DPPNPCPLNYKSHDLPSPCDHGIEDTIEFNRNWIIRKMENGECSCDNEH
MDSCPIESNENGDKNNFVSAQKADRKPYWVNPYLRGESRKRLVAKKRVK
RSHTSFPSFQVYHYNPYLMGSVHKTAVKKIGPYKPSHEKYM
*The underlined part is the His tag sequence on the vector.

In the present invention, the protein of the present invention also includes its conservative variant, which refers to the polypeptide formed by substituting up to 10, preferably up to 8, more preferably up to 5, and most preferably up to 3 amino acids with amino acids of similar or analogous properties, compared with the amino acid sequence of the protein of the present invention (SEQ ID NO: 1 and/or SEQ ID No: 2). These conservative variant polypeptides are preferably produced by amino acid substitutions according to Table 1.

TABLE 1

| | Representative | Preferred |
|---|---|---|
| Initial residue | substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

As used herein, “isolated” refers to a substance being separated from its native environment (if the substance is naturally occurring, its native environment is the natural environment). For example, the polynucleotides and polypeptides in the natural state within living cells are not isolated or purified, but the same polynucleotides or polypeptides are considered isolated and purified if they are separated from other substances coexisting in the natural state.

The polynucleotide of the present invention may be in the form of DNA or RNA. DNA form includes cDNA, genomic DNA, or synthetic DNA. DNA may be single stranded or double stranded. DNA may be a coding strand or a non-coding strand. The coding region sequence encoding mature polypeptides may be identical to the coding region sequences as set forth in SEQ ID NOs: 5 and 6, or may be degenerate variants thereof. As used herein, “degenerate variant” in the present invention refers to a nucleic acid sequence that encodes a protein with SEQ ID NOs: 1, 2, but differs from the coding region sequences as set forth in SEQ ID NOs: 5, 6.

The nucleotide sequences involved in the present invention are as follows:

Sj-SP-19 (SEQ ID NO: 5):
ATGGTGTACATGATAAAATATGACTCCACCCATGGAAAGTTTCAAGGTG
ATGTTTCGGTTGAGAACGGAAAACTTAATGTCAATGGAAGGCTTATATC
AGTTTACTGCGAGAGGGATCCATTGAACATACCATGGAACAAGGATGGT
GCTGAGTATGTTGTAGAGTCCACTGGAGTCTTCACTACAATTGATAAAG
CTCAAGCTCATATTAAAAACGATCGGGCTAAAAAAGTTATAATATCAGC
TCCCTCGGCAGACGCACCCATGTTTGTTGTTGGTGTGAATGAAAAGACT
TACGACAAGTCAATGTCTGTGGTTTCGAATGCATCGTGCACCACAAACT
GTCTAGCACCTCTAGCTAAAGTCATTAATGACAATTTTGAAATAGTTGA
AGGCCTTATGACTACTGTACACTCATTTACGGCTACGCAAAAGACCGTT
GATGGACCATCTTCAAAACTGTGGAGAGATGGTCGTGGGGCGTTTCAGA
ATATTATTCCAGCCTCCACTGGTGCTGCAAAGGCAGTGGGCAAAGTCAT
CCCTGCATTAAACGGAAAGTTGACAGGAATGGCTTTCCGGGTGCCTACA
GCGAATGTTTCAGTAGTTGACCTGACATGCAGATTGGGCAAAGGAGCTA
CCTACGATCAAATCAAGGCTGTGATCAAAGCAGCCGCAAATGGACCATT
AAAAGGCATCTTGGAATATACTGAAGATGAAGTTGTCAGCTCAGACTTT
ATTGGATGTACCAGTTCATCCATATTTGATGCAAAGGCTGGAATCTCTC
TCAACAACAATTTCGTGAAACTGGTTTCATGGTACGACAATGAATTCGG
CTACAGTTGCCGCGTGGTCGATCTCATCACGCATATGCATAGAGTCGAC
CATTCTTA
Sj-SP-489 (SEQ ID NO: 6):
ATGAACCAAATCAAACCTAGAATATTATTTCTGTTAGTGCTTTTAATTG
ATCTGTATGATCGAATATTAGCAAGTAATTATGATCAGTATATAGATAG
ATTGACAAATGATGGCAAATTATTATACGATGATTATATTAAACAGAAT
CCTAGTTTAGAATCAGCATTAGAACGATTATATACGCTACAACATCCAA
TTTTTCAAGAAGATTATCCAGGAAATTATGAGATTACTGATAAACAATG
GAATGCATTTCTAAATGAAATCGATCAAGCTAAATTAGGCAGACTGCAA
AACAATGATGCTGATAAACCAGAGATGACCTACTCAAATCTCGACAGAA
AATCGAATTATGAATTGTATCCAAATACAAATAATAATAATGACAAAGT
ATTTAAATGAACCTAGTTTAACAGAACGTCGAAATGAAATCGCCTATCA
AATCCACTGTGGGGTGAACATAAAGTTACTGGTGGTTCCAGTGAAACAG
GTCAATGGATAGATTATGCTTTATTAGGAGCTGGAGCACAAGATCTACT
TGATAATGAATCATCAGTTGATAATTTTAATCTTTCCAATGAAATAGAA
TCATCTCATATAGAGTCAAAAGTTGATAATTTACCAGCATATTGTGATC
CACCTAATCCTTGTCCATTAAATTATAAATCACATGATTTACCGTCACC
ATGTGATCATGGTATTGAAGATACTATCGAGTTTAATCGAAACTGGATA
ATAAGGAAAATGGAAAATGGTGAATGTTCATGTGACAATGAACATATGG
ATAGTTGCCCAATTGAATCAAATGAAAATGGAGACAAAAATAATTTTGT
TTCAGCACAAAAGGCGGATAGAAAACCATACTGGGTTAATCCATATCTC
CGGGGTGAAAGTCGAAAAAGGCTCGTAGCTAAGAAACGAGTAAAGCGTT
CACATACTTCTTTTCCATCTTTTCAGGTATACCATTACAATCCTTATCT
GATGGGTAGCGTTCATAAAACAGCAGTGAAAAAAATTGGACCATACAAA
CCATCCCATGAAAAATATATGTAA

The polynucleotides encoding the mature polypeptides of SEQ ID NO: 5, 6 includes: the coding sequence that encodes only the mature polypeptide; the coding sequence of the mature polypeptide and various additional coding sequences; the coding sequence of the mature polypeptide (and optional additional coding sequence) and the non-coding sequence.

The term “polynucleotide encoding a polypeptide” may be a polynucleotide that includes sequence encoding the polypeptide, or a polynucleotide that also includes additional coding and/or non-coding sequences.

The present invention also relates to variants of the above-mentioned polynucleotides, which encode polypeptides having the same amino acid sequence as those of the present invention, or fragments, analogs, and derivatives of such polypeptides. The variants of these polynucleotide can be naturally occurring allelic variants or non-naturally occurring variants; they can also be polynucleotide variants generated by using different codons to encode the same amino acids. These nucleotide variants include substitution variants, deletion variants, and insertion variants. As known in this field, an allelic variant is an alternative form of a polynucleotide that may involve a substitution, deletion, or insertion of one or more nucleotides, but does not substantially alter the function of the encoded polypeptide.

As used herein, the term “primer” refers to a general term for oligonucleotides that can serve as a starting point to synthesize DNA strands complementary to the template by pairing with the template and under the action of DNA polymerase. A primer can be natural RNA, DNA, or it can also be any form of natural nucleotides. A primer can even be a non-natural nucleotide such as LNA or ZNA. A primer is “substantially” (or “essentially”) complementary to a specific sequence on one strand of the template. A primer must be sufficiently complementary to one strand of the template to initiate extension, but the sequence of the primer does not need to be completely complementary to the sequence of the template. For example, adding a sequence that is not complementary to the template to the 5′ end of a primer whose 3′ end is complementary to the template still results in a primer that is substantially complementary to the template. As long as a sufficiently long primer can fully bind to the template, a non-completely complementary primer can also form a primer-template complex with the template, thereby enabling amplification.

The full-length nucleotide sequence of the protein of the present invention or fragments thereof may generally be obtained by PCR amplification, recombination or artificial synthesis methods. For PCR amplification, primers can be designed based on publicly available relevant nucleotide sequences, especially the open reading frame sequences, and commercially available cDNA libraries or cDNA libraries prepared using conventional methods known to those skilled in the art can be used as templates to amplify the relevant sequences. When the sequence is long, it is often necessary to perform two or more rounds of PCR amplification, and then splice the amplified fragments together in the correct order.

Once the relevant sequence is obtained, the recombination method can be used to obtain the relevant sequence in large quantities. This typically involves cloning it into a vector, transforming it into a cell, and then isolating the relevant sequence from the proliferated host cell by conventional methods.

In addition, the relevant sequence can also be obtained by artificial synthesis, especially when the fragment length is short. Generally, fragments with very long sequences can be obtained by first synthesizing multiple small fragments followed by performing ligation.

The method of amplifying DNA/RNA using PCR technology is preferably used for obtaining the genes of the present invention. The primers used for PCR can be appropriately selected based on the sequence information of the present invention disclosed herein, and can be synthesized using conventional methods. The amplified DNA/RNA fragments can be isolated and purified by conventional methods such as gel electrophoresis.

The present invention also relates to a vector containing the polynucleotide of the present invention, as well as a host cell generated by genetic engineering using the vector or the coding sequence of fusion proteins of the present invention, and a method for producing the protein of the present invention through recombinant technology.

The sequence of the present invention can be used to express or produce recombinant proteins through conventional recombinant DNA techniques. In general, the steps are as follows:
(1) Transforming or transducing a suitable host cell with a polynucleotide (or variant) encoding the protein of the present invention, or with a recombinant expression vector containing the polynucleotide;
(2) Culturing the host cell in a suitable medium;
(3) Isolating and purifying the protein from the medium or the cells.

The methods well-known to those skilled in the art can be used to construct expression vectors containing coding DNA sequences of the protein of the present invention and appropriate transcription/translation control signals. These methods include in vitro recombinant DNA technology, DNA synthesis technology, in vivo recombinant technology, etc. The DNA sequence can be effectively linked to appropriate promoters in the expression vector to guide mRNA synthesis. The expression vector also includes ribosome binding sites for translation initiation and transcription terminators.

In addition, the expression vector preferably contains one or more selectable marker genes to provide phenotypic traits for selecting transformed host cells, such as dihydrofolate reductase, neomycin resistance, and green fluorescent protein (GFP) for eukaryotic cell culture, or tetracycline or ampicillin resistance for Escherichia coli.

Vectors containing the aforementioned appropriate DNA sequences and appropriate promoters or control sequences can be used to transform appropriate host cells, enabling them to express proteins.

Host cells may be prokaryotic cells, such as bacterial cells; or lower eukaryotic cells, such as yeast cells; or higher eukaryotic cells, such as mammalian cells. Representative examples include: bacterial cells of Escherichia coli and Streptomyces; fungal cells such as yeast; plant cells; insect cells of Drosophila S2 or Sf9; animal cells of CHO, NSO, COS7 or 293 cells, etc.

Transformation of host cells with recombinant DNA can be carried out using conventional techniques well known to those skilled in the art. When the host is a prokaryote such as Escherichia coli, the competent cells capable of absorbing DNA can be harvested after the exponential growth phase and treated with CaCl₂) method, with the procedures being well known in the art. Another method is to use MgCl₂. If necessary, the transformation can also be carried out by electroporation. When the host is eukaryote, the following DNA transfection methods can be used: calcium phosphate co-precipitation method, conventional mechanical methods such as microinjection, electroporation, liposome encapsulation, etc.

The obtained transformant can be cultured by conventional methods to express the polypeptide of the present invention. Depending on the host cell used, the medium used in the cultivation may be selected from a variety of conventional culture media. Cultivation is carried out under conditions suitable for the growth of the host cell. When the host cells grow to an appropriate cell density, the selected promoter is induced by a suitable method (such as temperature conversion or chemical induction), and the cells are cultured for an additional period of time.

The protein in the above method may be expressed in the cell, or on the cell membrane, or secreted outside the cell. If necessary, the protein can be isolated and purified by various separation methods using its physical, chemical and other properties. These methods are well known to those skilled in the art. Examples of these methods include, but are not limited to, conventional renaturation treatment, treatment with a protein precipitant (salting-out method), centrifugation, osmotic lysis, sonication, ultra-centrifugation, molecular sieve chromatography (gel filtration), adsorption chromatography, ion exchange chromatography, high performance liquid chromatography (HPLC) and other liquid chromatography techniques and combinations of these methods.

The recombination method can be used to obtain the peptide sequence of the present invention in large quantities. This typically involves cloning it into a vector, transforming it into a cell, and then isolating the relevant peptides from the proliferated host cell by conventional methods.

In addition, chemical methods can be used to directly synthesize relevant peptide sequences.

### Pharmaceutical Composition

Due to the excellent activity of activating alveolar macrophages and/or anti-cancer properties of the polypeptides of the present invention, the polypeptides of the present invention (including wild-type, or their active fragments, or mutants within the range of retaining their polypeptide activity, or their pharmaceutically acceptable salts or esters), as well as pharmaceutical compositions containing the polypeptides of the present invention as the main active ingredient, can be used to activate alveolar macrophages and/or prevent and/or treat tumors.

The pharmaceutical composition of the present invention comprises a safe and effective amount of the polypeptide of the present invention and a pharmaceutically acceptable excipient or carrier. Among them, “safe and effective amount” refers to an amount of the compound sufficient to significantly improve the condition without causing serious side effects. Typically, the pharmaceutical composition contains 1-2000 mg of the polypeptides of the present invention, more preferably, contains 10-200 mg of the polypeptides of the present invention. Preferably, the “one dose” refers to a capsule or a tablet.

“Pharmaceutically acceptable carrier” refers to one or more compatible solid or liquid fillers or gel substances, which are suitable for human use, and must have sufficient purity and sufficiently low toxicity. “Compatibility” herein means that each component in the composition can be blended with the targeted inhibitor of the present invention and with each other, without significantly reducing the efficacy of the compound. Some examples of pharmaceutically acceptable carriers include cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween @), wetting agents (such as sodium dodecyl sulfate), colorants, flavoring agents, stabilizers, antioxidants, preservatives, pyrogen free water, and the like.

The representative administration methods of the polypeptide or the pharmaceutical composition of the present invention include but are not limited to: inhalation and parenteral (intravenous, intramuscular or subcutaneous) administration.

The composition for parenteral injection may comprise physiologically acceptable sterile aqueous or non-aqueous solutions, dispersions, suspensions or emulsions, and sterile powders for reconstitution into sterile injectable solutions or dispersions. Suitable aqueous and non-aqueous carriers, diluents, solvents or excipients include water, ethanol, polyol, and suitable mixtures thereof.

The composition for inhalation may comprise aerosols, sprays, etc.

The polypeptide of the present invention can be administered alone or in combination with other pharmaceutically acceptable compounds.

When administered in combination, the pharmaceutical composition further comprises one or more (2, 3, 4, or more) other pharmaceutically acceptable compounds. One or more of these other pharmaceutically acceptable compounds may be administered simultaneously, separately, or sequentially with the compounds of the present invention.

When using the pharmaceutical composition, a safe and effective amount of the polypeptide according to the present invention is administered to a mammal (e.g., human) in need of treatment, wherein the administration dosage is a pharmaceutically effective dosage. For a person with a body weight of 60 kg, the daily dose is usually 1-2000 mg, preferably 10-500 mg. Of course, the specific dosage should also consider factors such as the administration route and the patient's health status, which are all within the skill range of a skilled physician.

### Therapeutic Method

The present invention also provides a method for treating tumors, wherein a safe and effective amount of the active ingredient or pharmaceutical composition of the present invention is administered to a subject in need thereof, thereby treating the tumors.

### The Main Advantages of the Present Invention

(a) Strong anti-tumor effect: The FES and its effector protein Sj-SP-489 of the present invention can inhibit more than 90% of metastatic tumors in the lungs and liver.

(b) Anti-tumor effect on multiple tumors: It exhibits significant inhibitory effects on various tumors, including lung cancer cells, melanoma cells, leukemia, and lymphocytoma.

(c) Clear anti-tumor immune mechanism: The anti-tumor effects are exerted by activating alveolar macrophages and cytokines such as IL-1β secreted by them.

(4) Discovery of schistosome-derived effector proteins mediating anti-tumor effects: It is found that the secreted and excreted proteins of eggs, Sj-SP-489 and Sj-SP-19, exhibit anti-tumor effects equivalent to those of FES, thereby expanding the practical application and prospects of the present invention in the prevention and treatment of tumors.

The present invention is further explained below in conjunction with specific examples. It should be understood that these examples are only for illustrating the present invention and not intend to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturers. Unless otherwise stated, percentages and parts are calculated by weight.

### General Methods and Materials

1. Establishment of a Mouse Tumor Model Naturally Infected with Schistosoma japonicum (for Example 1)

On day 0, C57BL/6 mice were infected with 16 Schistosoma japonicum cercariae via the abdominal cavity. On day 40, 1×10⁶ B16/F10 or LLC tumor cells were intravenously injected into the mice through the tail vein. B16/F10 and LLC model mice were euthanized around day 55 and day 65, respectively, and samples were collected.

### 2. Isolation of Eggs and Preparation of Dead Eggs (for Example 2)

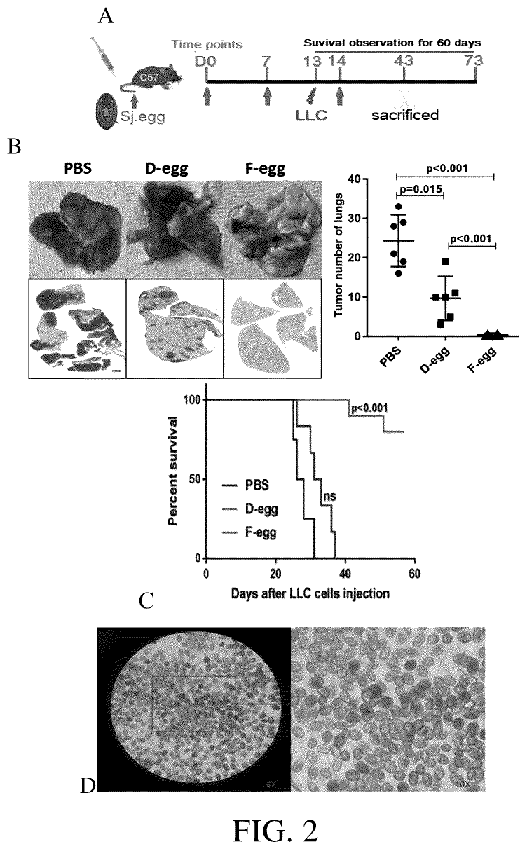

2.1 Isolation of fresh live eggs: (1) KM mice were infected with 40 cercariae via abdominal cavity; (2) The mice were euthanized on days 42-49, and their livers were isolated and washed several times with PBS. (3) The livers were homogenized with a grinder, then the homogenate was subjected to passing through 80 mesh and 150 mesh filters in order, and the filtrate was collected. (4) The filtrate was centrifuged at 4° C., 3000 rpm for 2 min, then the supernatant was discarded, and the precipitate was resuspended in 1.2% NaCl. The centrifugation step was repeated for 3 times. (5) The precipitate was resuspended in PBS, and added with trypsin, triple antibiotics, and DNAse I. The mixture was vortexed to homogenize, and then placed on a shaker at 37° C. and shaken at 200 rpm for 3 hours. (6) Centrifugation was conducted at 4° C., 50 g for 10 minutes, and the supernatant was discarded. (7) The precipitate was resuspended in 2 mL PBS and was slowly added to the upper layer of a 50% Percoll separation solution. Centrifugation was conducted at 4° C., 800 g for 5 min. (8) The supernatant was discarded. The precipitate was resuspended in PBS, then allowed to settle naturally for 10 minutes, and the supernatant was discarded. (9) The precipitate was resuspended in 2 mL PBS, then observed and counted under a microscope. A large number of fresh live eggs could be observed, with a clean background free of impurities and only a small amount of eggshell (FIG. 2 Panel D).

2.2 Preparation of dead eggs: Live eggs were boiled in boiling water for 1 hour, then centrifuged at 4° C., 300 g, for 5 minutes. The supernatant was discarded and resuspended in PBS, followed by natural sedimentation for 10 minutes. This process was repeated for three times.

2.3 Animal model: On day 0, 5000 live eggs were injected into C57BL/6 or NOD-SCID mice through the tail vein, while the mice of control group were injected with 5000 dead eggs or 200 μL PBS. The second injection was conducted on day 7. On day 13, the mice were injected with 1×10⁶ B16/F10 or LLC cells. The third injection was conducted on day 14. Around day 33 and day 43, the B16/F10 and LLC model mice were euthanized respectively, and samples were collected to observe the number of tumors in the lungs and liver of the mice, or the survival time of the mice was observed.

### 3. Experimental Method for Verifying Whether the Anti-Tumor Effect Depends on T and B Cells (for Example 3)

Egg preparation was performed as described above. B16/F10 and LLC cells were cultured in RPMI 1640 medium or high glucose DMEM medium containing 10% FBS and dual antibiotics, respectively, in a cell culture incubator at 37° C. with 5% CO2. According to the method and time points described in section 2.3 above, 6-week-old male NOD-SCID mice housed in housed in SPF grade animal facility were injected with the eggs, as well as B16/F10 and LLC cells. The number of tumors in the lungs and liver of mice was observed, or the survival time of mice was observed.

### 4. Preparation of FES (or DES) (for Example 5)

(1) The eggs used for FES preparation were derived from New Zealand rabbits infected with Schistosoma japonicum. The limbs of the New Zealand rabbit were fixed on the bracket, the abdominal hair was removed and the skin was moistened with water. 800-1000 cercariae were placed on a coverslip, which were used for infection of the rabbit through the abdominal skin. After the cercariae penetrated the rabbit's skin for about 10 minutes, the coverslip was removed. After 42 days of infection with cercariae, the rabbits were euthanized and the eggs of Schistosoma japonicum were isolated using the method described in section 2.1 above.

(2) The live (or dead) eggs were inoculated in a 12 well plate at a density of 200,000 eggs per well, and 3 mL of 1640 medium containing antibiotics was added to each well. (2) 2 mL of supernatant was collected every 24 hours accompanied by 2 mL of fresh culture medium was supplemented. (3) The collected supernatant was centrifuged at 4° C. and high speed for 10 minutes. This centrifugation step was repeated twice, and the supernatant was collected. (4) The supernatant was filtered with a 0.22 μm filter and concentrated to 1/10 of its original volume using a 3 KD ultrafiltration tube, then the concentrated product was stored in a −80° C. refrigerator.

### 5. Preparation of Single Cell Suspension of Lung Tissue (for Example 6)

(1) Mice were anesthetized with isoflurane and euthanized by exsanguination via the eyeballs. (2) The lungs were isolated and washed several times with PBS, then placed into dissociation tubes. The dissociation enzymes provided in the Miltenyi lung tissue dissociation kit were added. (3) The lungs were homogenized using a grinder. The homogenate was placed in a constant temperature shaker at 37° C. and subjected to digestion at 200 rpm for 30 minutes. (4) The tissue digestion solution was filtered using a 40 μm cell strainer and the filtrate was collected. (5) The filtrate was centrifuged at 4° C., 300 g, for 10 minutes, then the supernatant was discarded, and the precipitate was resuspended in PBS. The centrifugation step was repeated for twice. After resuspension of the precipitate with PBS, a single cell suspension of lung tissue was obtained.

6. Isolation and Cultivation of Primary AMs from Mice (for Example 6)

(1) The bronchoalveolar lavage fluid was heated to 37° C. and maintained at that temperature. (2) Mice were anesthetized with isoflurane and euthanized by exsanguination via the eyeballs. (3) The lungs and trachea of the mice were exposed, and a “V”-shaped upward incision was made above the trachea using scissors. (4) A 1 mL syringe was inserted into the trachea and fixed with thread. (5) 700 μL of bronchoalveolar lavage fluid was aspirated and injected into the lungs, then slowly withdrawn, and approximately 500-600 μL of alveolar lavage fluid could be collected each time. (6) The lavage process was repeated for 15-20 times, and 10 mL of alveolar lavage fluid could be collected from each mouse. (7) The lavage fluid was filtered through a 70 μm cell strainer and transferred to a new 15 mL centrifuge tube. (8) The lavage fluid was centrifuge at 4° C., 300 g, for 7 min, and the supernatant was discarded. (9) The residual red blood cells were lysed using red blood cell lysis buffer. The centrifugation was conducted again, and the supernatant was discarded. (10) The precipitate was resuspended in 1640 complete culture medium. By staining with trypan blue, the cells were subjected to the observation of the viability and counting, then the cells were inoculated onto a culture plate for cultivation. (11) Cultivation of primary AMs: the primary AMs were inoculated in a 24 well plate and cultured in RMPI 1640 complete medium. After 4 hours, the culture medium was removed, the suspended cells were washed away with PBS, and fresh culture medium was added.

### 7. Preparation of Single-Cell Sequencing Samples (for Example 7)

(1) On day 0, the live eggs, dead eggs or PBS were injected into C57BL/6 mice, and the injection was repeated on day 7, with 5 mice in each group. (2) On day 12, mice were euthanized, lungs were isolated, and single cell suspensions were prepared using the Miltenyi tissue dissociation kit. (3) AMs were labeled with FITC-CD11c, PE-F4/80, and APC Siglecf flow cytometry antibodies. (4) High purity AMs were sorted by flow cytometry, with 100,000 cells sorted from each mouse. The AMs sorted from the same group of mice were pooled into one tube. (5) After washing the cells twice with DPBS, they were handed over to a biotechnology company for single-cell sequencing and library construction.

### 8. Analysis of Single-Cell Sequencing Data (for Example 7)

(1) The Seurat package in R language was used to read the expression matrices analyzed by CellRanger, and data quality control, batch effect removal, normalization, dimensionality reduction, and clustering analysis were performed. The SingleR package was used to identify cell types. (2) Calculation of average gene expression levels: The colMeans function was used to calculate the average expression level of the corresponding gene sets in each cell, and box plots were generated. (3) Gene set enrichment analysis: The AddModuleScore function built into the Seurat package was used to score the gene sets, and the scoring results were visualized through empirical cumulative distribution function (ECDF) and violin plots.

### 9. Detection of IL-1β in Mouse Serum (for Example 7)

(1) Mouse blood was collected from eyeball blood collection, centrifuged at 5000 rpm for 15 minutes, and the supernatant was collected. The centrifugation was repeated once, and the collected serum was stored in a −80° C. refrigerator for later use. (2) The expression level of IL-1β in mouse serum was detected by using the mouse IL-1β ELISA detection kit provided by Thermo Company.

### 10. IL-1β In Vivo Neutralization Experiment (for Example 7)

FES was injected on days 0, 3, 6, and 9, B16-F10 cells were injected on day 7, and mice were treated on day 25. To inhibit IL-1β, anti-IL-1β antibody (B122) was injected intraperitoneally into mice at a dose of 0.625 mg/kg per injection, once every 3 days. Control mice were injected with an equal amount of IgG antibody.

### 11. Inactivation Treatment of Active Substances in FES (for Example 8)

(1) Protein digestion: Proteinase K was added to FES to a final concentration of 100 μg/mL, then subjected to incubation in a 56° C. water bath for 1 hour, and placed in boiling water for 10 minutes to inactivate proteinase K. (2) Digestion of DNA and RNA: DNase or RNase was added to FES to a final concentration of 50 mg/mL or 25 mg/mL, and subjected to digestion at room temperature for 2 hours.

### 12. Preparation of FES Containing Proteins of Different Molecular Weights (for Example 8)

(1) The filtered culture supernatant was placed in an ultrafiltration centrifuge tube with a molecular weight cutoff of 50 kDa, and the filtrate (i.e. FES containing proteins with molecular weight<50 kDa) was collected. (2) The filtrate was placed in an ultrafiltration centrifuge tube with a molecular weight cutoff of 30 kDa, and the concentrate (i.e., FES containing proteins with molecular weights<50 kDa and >30 kDa, designated as f4) was collected; Similarly, other FES fractions with different molecular weight ranges (f1, f2, f3, and f5) were prepared as follows: i.e., f1<3 kDa; 3 kDa<f2<10 kDa; 10 kDa<f3<30 kDa; 30 kDa<f4<50 kDa and f5>50 kDa.

### 13. Expression, Purification, and Identification of His-Tagged Proteins (for Example 8)

(1) The gene sequence information was acquired from the NCBI website based on the protein name information of the f4 fraction obtained through mass spectrometry analysis. (2) DNA fragments were synthesized by a company and ligated into the pET28a vector. (3) The recombinant vector was transformed into BL21 (DE3) and protein expression was induced with IPTG. (4) Escherichia coli was lysed and the supernatant and precipitate (containing inclusion bodies) were collected. (5) The precipitate of bacterial lysate was solubilized with inclusion body solubilization buffer containing urea and purified using a purification resin. (6) The collected protein eluate was put into a refolding bag and subjected to refolding with refolding solution. (7) The refolded proteins were transferred into an ultrafiltration tube for concentration, and the protein concentration was determined using the BCA method. (8) The protein expression was identified using SDS-PAGE electrophoresis.

### 14. Screening of His-Tagged Proteins (for Example 8)

(1) MH-S cells in logarithmic growth phase were inoculated in 96 well cell culture plates. (2) The purified protein at a final concentration of 10 mg/μL was added to each well and the plates were incubated in a constant temperature incubator for 24 hours. (3) The culture supernatant was collected and the IL-1β protein concentration was detected using ELISA method. (4) The culture cells were collected and the expression levels of genes such as Il1b in cells were detected.

### Example 1 Schistosoma japonicum Infection Inhibits the Formation of Lung Metastatic Tumors

Cercariae of Schistosoma japonicum were prepared by artificially infecting its intermediate host, snails, and then used to infect natural hosts. Natural definitive hosts include humans and suitable animal hosts such as mice, rabbits, and water buffaloes. Cercariae can infect the host through the skin, and after entering the body, they migrate through the intrabody pathway to reach the mesenteric veins, where they develop into adults and lay eggs. The eggs produced are distributed along the mesenteric veins and portal venous return system, and deposit in the liver and intestinal wall tissues.

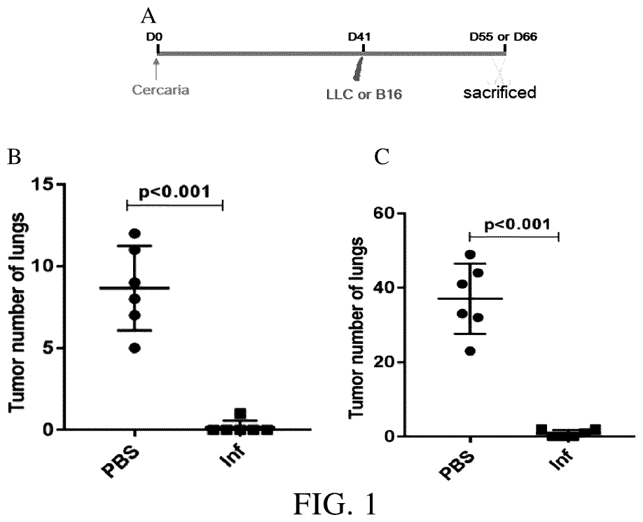

In the lung metastatic tumor model of lung adenocarcinoma LLC cells, mice in the infected group formed an average of 0.2±0.4 tumor foci in the lungs, which was significantly fewer than the 8.7±2.6 tumor foci in the uninfected (PBS) control group; In the B16 tumor model, mice in the infected group formed 0.8±1.0 tumor foci in the lungs, which was significantly fewer than the 37.0±9.4 tumor foci in the uninfected control group (FIG. 1). Therefore, compared with uninfected mice, schistosome infection can reduce the number of lung metastatic tumors in the two tumor cell models by 98.1% and 97.7%, respectively (P<0.001).

### Example 2 Schistosoma japonicum Eggs Inhibit the Formation of Lung Metastatic Tumors in Mice

Live eggs (F-egg), boiled and inactivated dead eggs (D-egg), and PBS were respectively injected into C57BL/6 mice through the tail vein, and the injected eggs deposited in the lungs, causing pulmonary egg granulomas. Subsequently, LLC tumor cells were injected to form lung metastatic tumors.

The results showed that among the 6 mice in the F-egg group, 4 mice had no tumor formation, while the other 2 each had one tumor focus. In contrast, the number of tumors in the D-egg control group mice was 9.7±5.6, and in the PBS group it was 24.3±6.6. Thus, compared with the D-egg group and PBS group, the F-egg group reduced the number of tumors by 96.6% and 98.6%, respectively (FIG. 2 Panel A, Panel B, P<0.001).

Moreover, F-eggs significantly prolonged the survival time of mice. 80% of the F-egg group mice (8 mice) remained alive at the end of the observation period (60 days), while all mice in the D-egg group and PBS group died within 37 and 31 days, respectively (FIG. 2 Panel C).

### Example 3 The Anti-Tumor Effect Mediated by Live Eggs is Independent of T and B Cells

To investigate whether the above anti-tumor activity depends on T and B cells, the inventors conducted a similar experiment using NOD-SCID mice, which lack mature T and B cells.

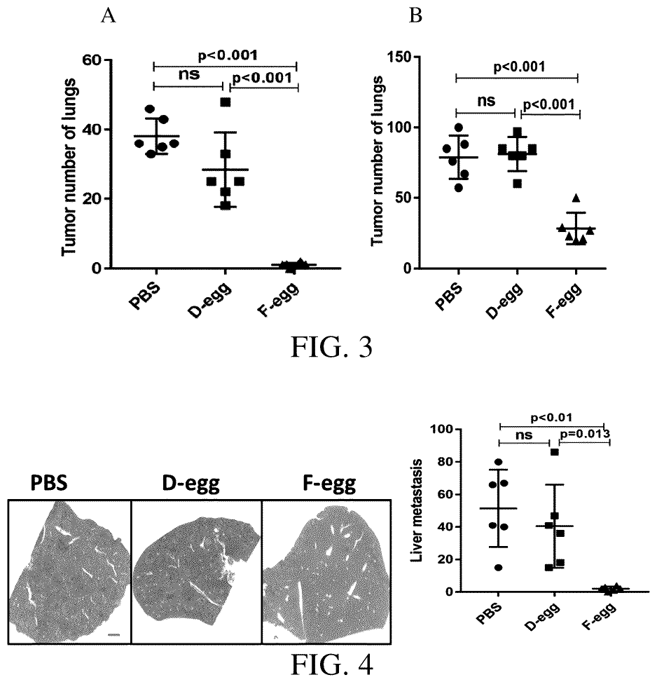

The results showed that in this immunodeficient mouse model, the number of tumors in the F-egg group (1±0.6) decreased by 96.5% and 97.4%, respectively, compared to the D-egg group (28.5±10.7) and PBS group (38.2±5.1) (P<0.001) (FIG. 3 Panel A). In addition, the inventors conducted similar experiments using the more invasive B16 melanoma cell line. The results showed that in the NOD-SCID mouse model, F-eggs significantly inhibited lung metastatic tumors, with a reduction of 64.1% and 65.1% in tumor number compared to the D-egg group and PBS group, respectively (FIG. 3 Panel B). These results indicate that the anti-tumor effect mediated by F-eggs is independent of T and B cells.

### Example 4 Inhibitory Effect of Schistosoma japonicum Eggs on Tumors in Distant Organs

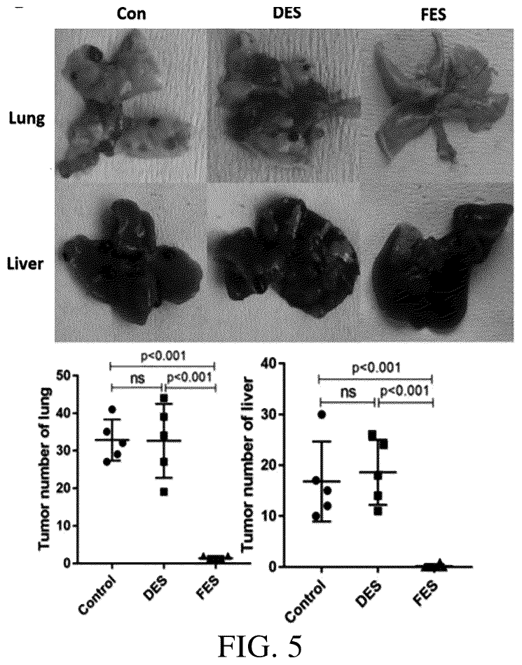

In the B16 cell metastatic tumor model, in addition to the formation of metastatic tumors in the lungs, macroscopically visible melanoma metastatic tumors can also form in the liver. The experimental procedure was the same as described previously. The eggs injected through the tail vein deposited in the lungs, and the F-eggs deposited in the lungs could significantly inhibit melanoma metastatic tumors in the liver. In the NOD-SCID mouse model, the number of liver tumors in the F-egg group (2±1.4) decreased by 96.1% and 95.1%, respectively, compared to the PBS group (51.5±23.8) and the dead egg control group (40.5±25.6) (P<0.01) (FIG. 4). In addition, as shown in FIG. 1, in the mouse model of natural Schistosoma japonicum infection, female worms produced a large number of eggs, which mainly deposited in the liver and intestinal wall tissues. The eggs deposited in the liver and intestinal wall tissues could exert a potent inhibitory effect on lung metastatic tumors. These results indicate that the Schistosoma japonicum eggs can exert a potent inhibitory effect on tumors in distant organs in vivo.

### Example 5 Anti-Tumor Effect Mediated by Secretions and Excretions of Eggs

As mentioned above, eggs deposited in the lungs or liver can exert inhibitory effects on metastatic tumors in distant organs. This result led the inventors to hypothesize that the anti-tumor effect of eggs may be mediated by their secretions and excretions. Therefore, the inventors prepared concentrated serum-free live fertile egg culture supernatant (FES) and dead egg culture supernatant (DES). These supernatants were injected into mice via tail vein on days 0, 3, 6, and 9, and B16 tumor cells were injected on day 7. Samples were collected on day 27.

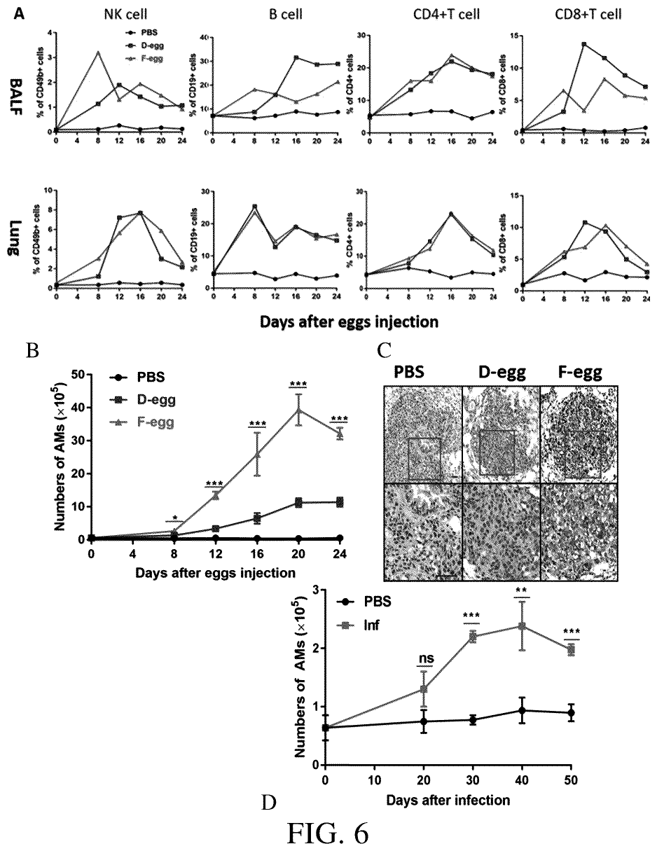

The results showed that mice in the FES group formed 1.4±0.5 tumor foci in their lungs, which was significantly fewer than the 32.6±9.9 tumor foci in the DES group and the 32.8±5.5 tumor foci in the culture medium control group (P<0.001) (FIG. 5). In the liver, the FES group had an average of 0.2±0.4 metastatic foci, while the DES group had 18.6±6.4 liver metastatic foci, and the egg culture medium control group had 16.8±7.9 liver metastatic foci. Therefore, the number of liver metastatic foci in the two control groups was significantly higher than that in the live egg supernatant group (P<0.001) (FIG. 5). The above results indicate that the secretions and excretions of eggs exert an anti-tumor effect with potency equivalent to that of live eggs, and suggest that the anti-tumor effect mediated by eggs is mediated and exerted through their secretions and excretions.

### Example 6 The Cellular Mechanism of the Anti-Tumor Effect Mediated by Live Eggs is the Activation of Alveolar Macrophages

To elucidate the cellular mechanism of the anti-tumor effect mediated by live eggs in the lungs, the inventors analyzed the composition of immune cells in the lungs and bronchoalveolar lavage fluid.

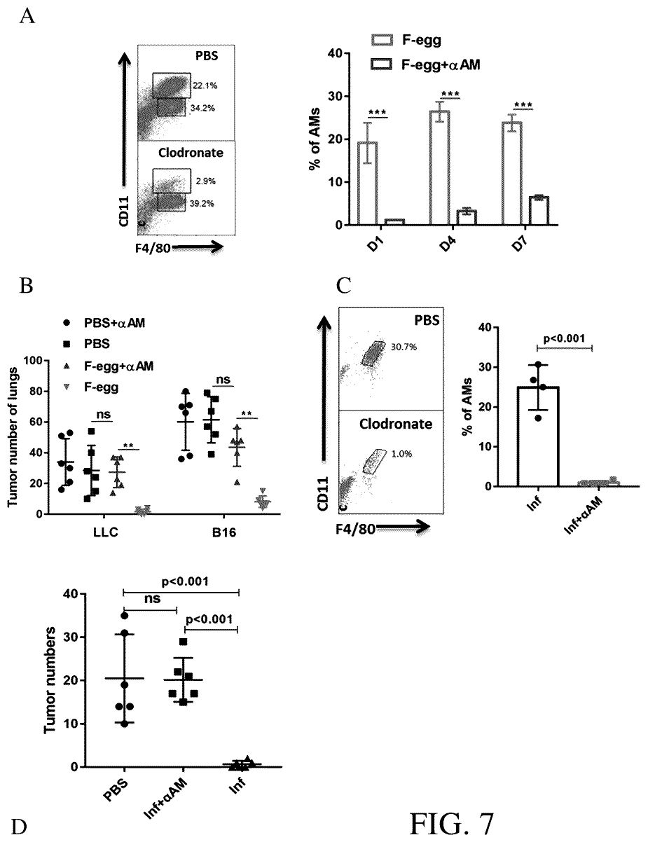

The results showed that in mice injected with 5,000 live eggs or dead eggs, the populations of CD4⁺T cells, CD8⁺T cells, NK cells, and B cells in their lungs and bronchoalveolar lavage fluid were significantly altered compared to the PBS group. However, there were no significant differences in these cell populations between the F-egg group and the D-egg group (FIG. 6 Panel A), while the number of alveolar macrophages (AMs, F4/80⁺, CD11c⁺, Siglec-F⁺) in the F-egg group was significantly higher than that in the D-egg group (FIG. 6 Panel B). Through immunohistochemical analysis, alveolar macrophages were found to infiltrate into tumor nodules (FIG. 6 Panel C). In addition, alveolar macrophages in the lungs were also elevated in the mouse model of natural infection (FIG. 6 Panel D). These results suggest that alveolar macrophages may be associated with F-egg mediated anti-tumor effects.

Subsequently, the inventors conducted functional studies on F-egg induced alveolar macrophages. Clodronate disodium liposomes were administered by tracheal instillation to deplete mouse alveolar macrophages without affecting other types of macrophages.

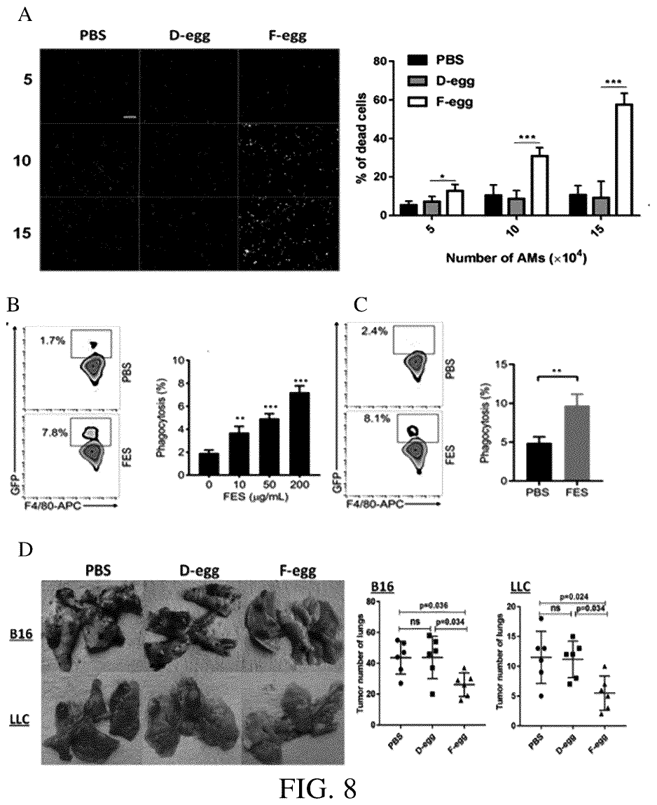

The results showed that after treatment with the depleting agent, the number of alveolar macrophages in the bronchoalveolar lavage fluid decreased by 90% (FIG. 7 Panel A). However, mice with depleted alveolar macrophages lost F-egg mediated anti-tumor activity: in B16 and LLC tumor cell models, the numbers of lung metastatic tumors in the F-egg+AMs depletion group were 43.5±12.3 and 27.3±10.0, respectively, which showed no significant difference from that in the PBS control group (P>0.05) (FIG. 7 Panel B). The inventors also obtained similar results in the natural infection model, where the inhibitory effect on lung metastatic tumors in infected mice was almost completely eliminated after alveolar macrophage depletion (FIG. 7 Panel C, Panel D). These results suggest that the anti-tumor effect medicated by eggs is dependent on alveolar macrophages.

In order to verify the anti-tumor effect of alveolar macrophages (Ams) induced by live eggs (F-eggs), the inventors isolated alveolar macrophages from mice in the egg-treated group and co-cultured them with tumor cells.

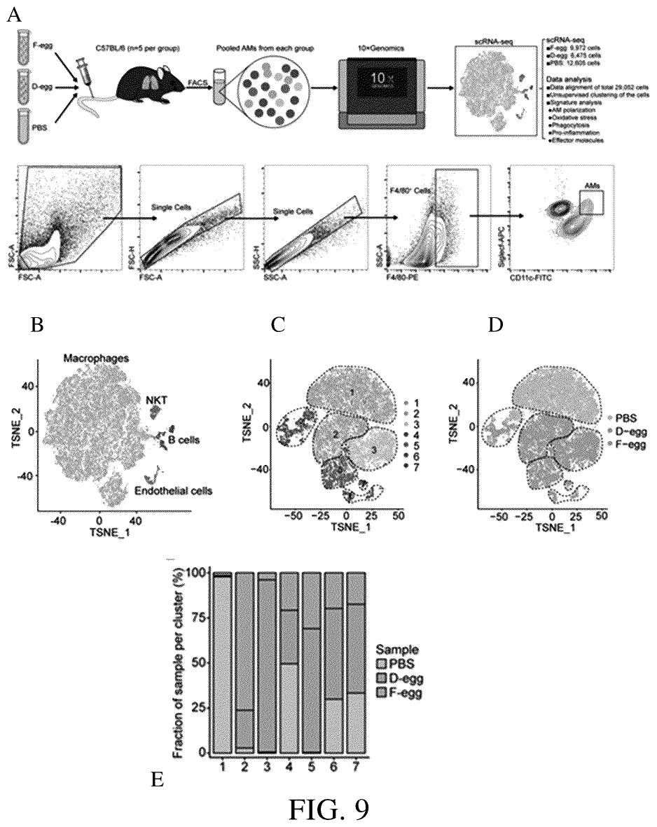

The results showed that the proportion of tumor cell death in the live egg group was significantly higher than that in the control group, and this effect was dependent on the concentration of co-cultured macrophages (FIG. 8 Panel A); In addition, the activity of FES activated AMs in phagocytosis of tumor cells was detected. The inventors constructed B16-GFP/Luc stably transfected cells, then co-cultured the FES activated AMs cell line (MH-S cells) with B16-GFP/Luc cells, and detected the percentage of GFP⁺F4/80⁺double positive cells in the total F4/80⁺ cells by flow cytometry. The results showed that the proportions of macrophages phagocytosing B16-GFP/Luc tumor cells in the control group and the three experimental groups were 1.9±0.3%, 3.7±0.5%, 4.9±0.4%, and 7.2±0.5%, respectively, indicating that the activated macrophages significantly enhanced their phagocytic ability towards B16-GFP/Luc tumor cells (FIG. 8 Panel B). At the same time, a similar experiment was conducted using FES activated AMs isolated from mice in vivo. The results showed that the phagocytic ratios of primary AMs towards tumor cells in the control group and FES group were 4.9±0.8% and 9.7±1.5%, respectively (P<0.01) (FIG. 8 Panel C). The above results indicate that FES activated AMs have enhanced their phagocytic activity towards tumor cells.

In addition, the inventors applied the method of cell reinfusion to verify the in vivo anti-tumor effect of alveolar macrophages induced by eggs. First, the inventors injected tumor cells through the tail vein on day 0, and on days 7, 10, and 13, transfused 5×10⁵ alveolar macrophages isolated from the mouse schistosome egg lung model into the mice through the trachea. Then, the mice were observed and samples were collected at appropriate times.

The results showed that in the LLC model, the number of lung tumors in mice reinfused with alveolar macrophages from the live egg group was reduced by 52.2% and 50.7% compared with that in the PBS group and dead egg group, respectively (FIG. 8 Panel D). In the B16 model, the number of tumors in the live egg group was reduced by 40.1% and 40.3% compared with that in the PBS group and dead egg group, respectively (FIG. 8 Panel D). This result indicates that the reinfusion of alveolar macrophages induced by live eggs into tumor bearing mice exerts anti-tumor effects.

### Example 7 Identification of the Phenotype and Anti-Tumor Effector Molecules of FES Activated AMs

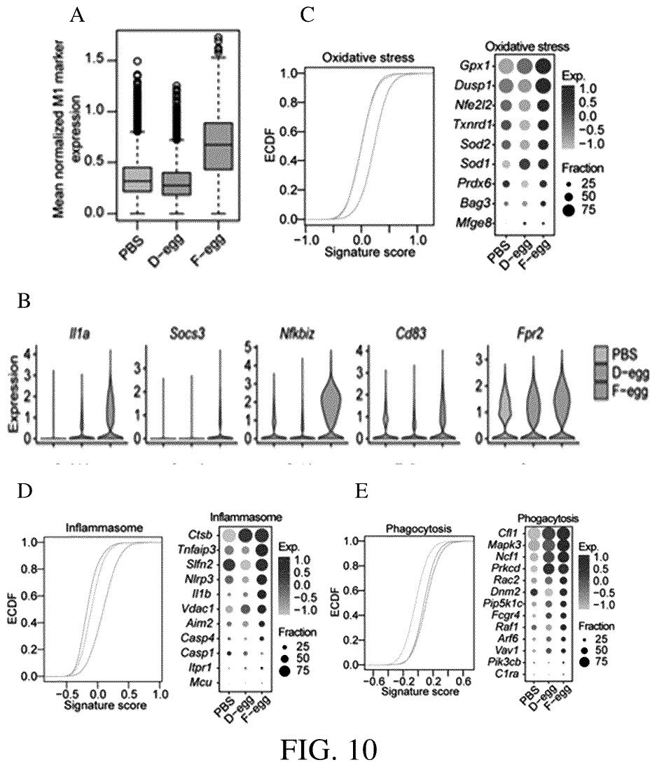

In order to elucidate the phenotype and anti-tumor effector molecules of AMs activated by eggs or FES thereof at the single-cell level, the inventors conducted single-cell transcriptome sequencing analysis on the activated AMs. First, the inventors injected F-eggs, D-eggs, and PBS into mice through the tail vein in two separate doses. Using flow cytometry sorting technology, AMs (F4/80⁺, CD11c⁺, SiglecF⁺) in lung tissues were sorted out (FIG. 9 Panel A) and subjected to 10× Genomics single-cell transcriptome sequencing. The inventors obtained data from 29,052 cells, including 12,605 cells from the PBS group, 6,475 cells from the D-egg group, and 9,972 cells from the F-egg group, with an average of 8,306 UMI and 2,531 genes detected per cell.

After reintegration, dimensionality reduction, and tSNE clustering of the data, the cells were divided into 7 subclusters (FIG. 9 Panel B, Panel C), with distinct cell sample compositions across the subclusters. The cells of subcluster 1 were mainly composed of cells from the PBS group, accounting for 97.7%; the cells of subcluster 2 were mainly composed of cells from the D-egg group, accounting for 76.2%; the cells of subcluster 3 were mainly composed of cells from the F-egg group, accounting for 95.5%; the cells of subcluster 5 were composed of cells derived from the D-egg and F-egg groups, accounting for 31.0% and 68.8%, respectively; and the other three subclusters were composed of cells from all three sample groups (FIG. 9 Panel D). As described above, AMs activated by F-eggs exhibit anti-tumor effects. Therefore, the F-egg group cells with anti-tumor effects were mainly distributed in two subclusters: subcluster 3 and subcluster 5.

### (1) Analysis of the Polarization Phenotype of AMs

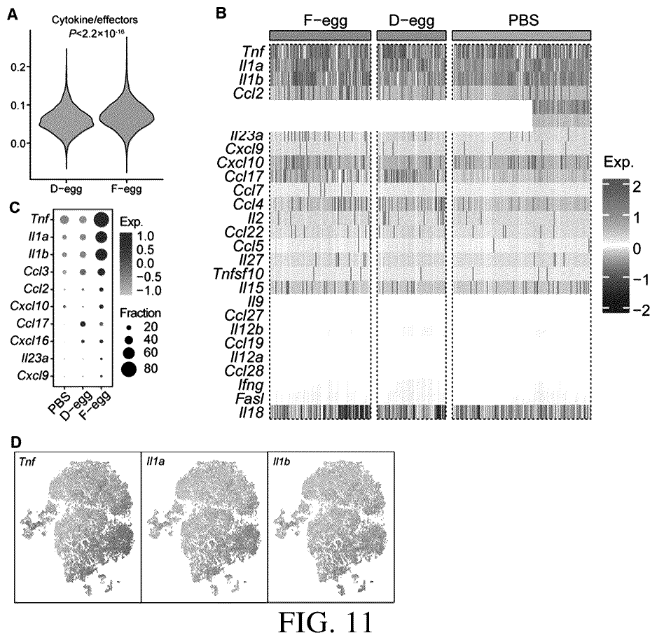

M1-type macrophages highly express cytokines such as TNF-α, IL-1β, IL-12, IL-6, and COX-2, which exert anti-tumor effects; M2-type macrophages highly express cytokines such as IL-10, IL-13, and TGF-β, which promote angiogenesis, tumor invasion, and metastasis. Given the important role of M1-type macrophages in anti-tumor activity, the inventors identified and analyzed the polarization phenotype of AMs in each sample. The inventors first selected some marker genes of M1-type macrophages, then calculated the average expression levels of the entire gene set among different samples and subclusters, and visualized the results using box plots. The results showed that AMs in the F-egg group exhibited high expression of M1-type marker genes, such as Il1a and Nfkbiz (FIG. 10 Panel A, Panel B).

### (2) Enrichment Analysis of Anti-Tumor Immunity Related Functions of AMs

The anti-tumor effects of macrophages involve multiple mechanisms, including phagocytosis, ROS, inflammasomes, etc. More and more studies suggest that ROS and inflammasomes have anti-tumor effects. Therefore, the present inventors performed functional enrichment analysis on gene sets related to phagocytosis, oxidative stress, and inflammasomes, and visualized the gene set enrichment and gene expression patterns through ECDF and dot plots. The results showed that the gene set characteristic of oxidative stress was significantly enriched in AMs from the F-egg group, including Gpx1 and Dusp1 encoding core enzymes of the oxidative stress pathway, and Sod1 and Sod2 encoding antioxidant enzymes (FIG. 10 Panel C). In addition, AMs derived from the F-egg group highly expressed inflammasome-related gene sets, such as the adaptor molecules Nlrp3, Aim2, and the downstream molecule Casp4 (FIG. 10 Panel D). Phagocytosis is one of the important ways in which macrophages exert anti-tumor effects. The genes expressed by AMs from the F-egg group were significantly enriched in phagocytic functions, and these genes include Fcgr4 encoding a phagocytic receptor and Prkcd encoding protein kinase C (FIG. 10 Panel E).

### (3) Identification of F-Egg Mediated Anti-Tumor Effector Molecules

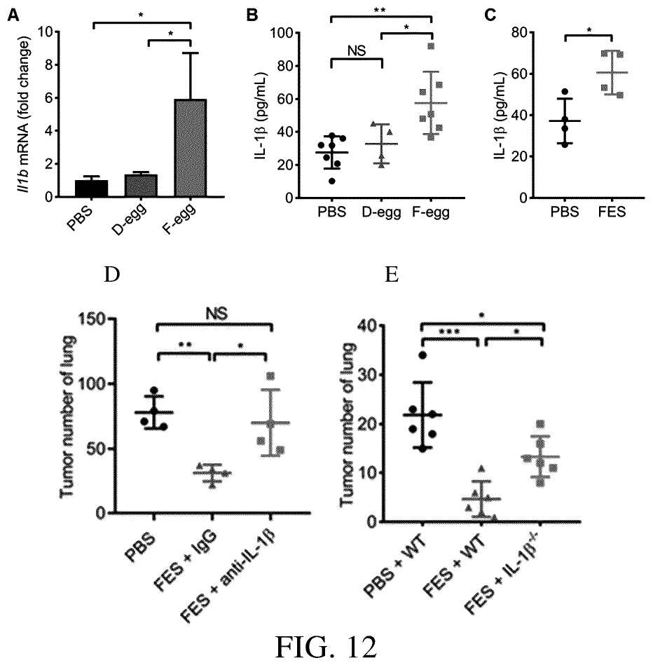

In order to identify the effector molecules through which AMs exert anti-tumor effects, the inventors used single-cell sequencing data to perform gene set scoring for the positive regulation of cell killing ability based on the GO dataset. The scoring results showed that the gene set score of F-egg group cells was significantly higher than that of D-egg group (FIG. 11 Panel A), indicating that F-egg activated AMs have stronger killing ability. According to existing literature reports, the inventors identified some cytokines with anti-tumor effects, such as Tnf, Il1b, Ccl2, Cxcl16, Il12b, Ifng, etc. Through heatmap and dot plot analyses, the inventors found that the F-egg group cells significantly highly expressed genes such as Tnf, Il1a, and Il1b (FIG. 11 Panel B, Panel C). The t-SNE plots showed that cells in subcluster 3 and subcluster 5, where the F-egg group cells were predominant, highly expressed Tnf, Il1a, and Il1b (FIG. 11 Panel D).

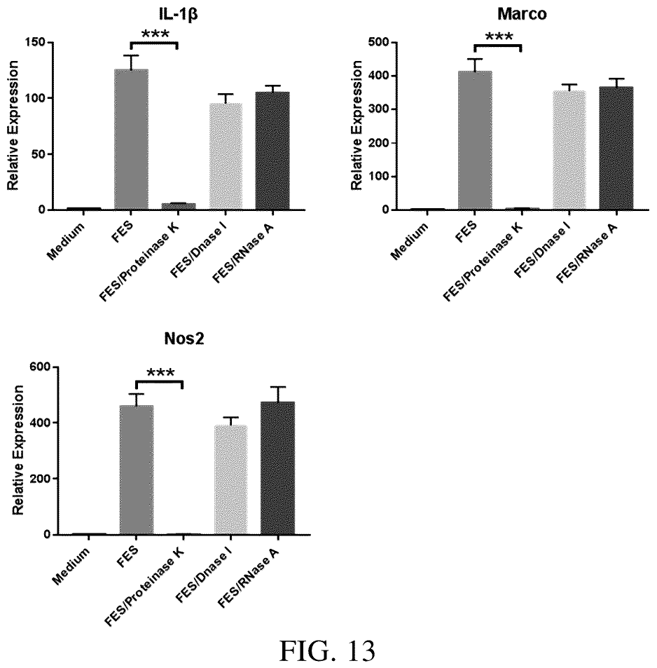

The expression of IL-1β was significantly increased in F-egg activated AMs. To investigate the role of IL-1β in the anti-tumor effect of egg activated AMs, the inventors isolated primary AMs activated by eggs. The qPCR detection showed that the expression level of Il1b in AMs from the F-egg group was increased by 5.93±2.78 folds (FIG. 12 Panel A). Then, the inventors detected the expression levels of IL-1β in the serum of each group of mice using ELISA method. The serum IL-1β levels in the PBS group, D-egg group, and F-egg group were 27.51±9.72 pg/mL, 32.67±11.76 pg/mL, and 57.54±18.97 pg/mL, respectively. F-eggs could significantly increase the IL-1β levels in the serum of mice (FIG. 12 Panel B). In addition, the inventors detected the level of IL-1β in the serum of mice injected with FES, and the results showed that FES could increase the level of IL-1β in the serum of mice (FIG. 12 Panel C). The above research results indicate that live eggs and FES upregulate the expression of IL-1β in AMs.

Next, the inventors investigated the role of IL-1β in the anti-tumor effect of egg activated AMs. The inventors employed a method of inhibiting IL-1β function, including using an IL-1β neutralizing antibody (B122) and IL-1β knockout mice, to detect whether the anti-tumor effect of FES activated AMs would change after inhibiting or knocking out IL-1β in mice. First, the inventors administered an intraperitoneal injection of IL-1β antibody (B122) to inhibit IL-1β in mice, while simultaneously injecting FES and B16 tumor cells. The results showed that the numbers of lung metastatic tumors in the PBS group, FES+IgG group, and FES+B122 antibody group were 78.0±12.38, 29.0±6.2, and 70.7±26.8, respectively (FIG. 12 Panel D). This result indicated that after inhibiting IL-1β (FES+B122 group), the anti-tumor effect of FES activated AMs was essentially eliminated, and the number of lung tumors was similar to that of the PBS control group. In addition, the inventors conducted further experiments using IL-1β knockout mice, and the results showed that the numbers of lung metastatic tumors in the mice of PBS+WT group, FES+WT group, and FES+IL-1β^{-/-} group were 21.8±6.6, 4.7±1.5, and 13.3±4.2, respectively (FIG. 12 Panel E). In summary, the inventors further demonstrated the important role of IL-1β in the FES mediated anti-tumor effects through related experiments on inhibiting or knocking out IL-1β in mice.

### Example 8 Identification of Schistosome-Derived Molecules Mediating Activation of Alveolar Macrophages and Anti-Tumor Effects

Previous studies have demonstrated the anti-tumor effect mediated by live Schistosoma japonicum eggs, which is achieved by activating AMs into M1-type and promoting their high expression of effector molecules such as IL-13. Further research has shown that the activation of AMs and the subsequent anti-tumor effect are mediated by the secretions and excretions of eggs (i.e., live fertile egg culture supernatant, FES). However, the key schistosome-derived molecules in FES that are responsible for activating AMs and exerting anti-tumor effects remain unclear. In this regard, the inventors conducted the following research:

### (1) the Effective Substance for Activating Macrophages in FES is Protein

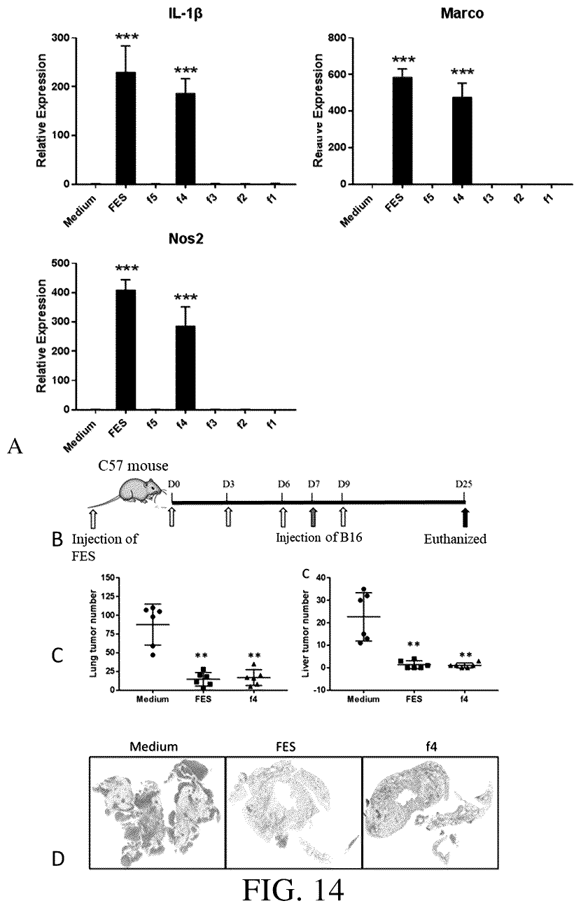

FES has complex components, and potential substances that may exert activation effects include DNA, RNA, and proteins, etc. In this regard, the inventors used DNase I, RNase A, and proteinase K to separately digest DNA, RNA, and proteins in FES, and observed the activation effect of the digested FES on the alveolar macrophage cell line (MH-S). The results showed that FES could activate MH-S and significantly upregulate the expression of Il1b mRNA and two other M1 markers (i.e. Marco and Nos2), while treatment with DNase I or RNase A did not affect FES's ability to activate MH-S. However, FES treated with protease lost the ability to activate MH-S for high expression of Il1b, Marco, and Nos2 mRNA (FIG. 13). This result indicates that the effective active component for activating MH-S in FES is protein.

(2) the Molecular Weight of Proteins for Activating MH-S in FES Ranges from 30 to 50 kDa

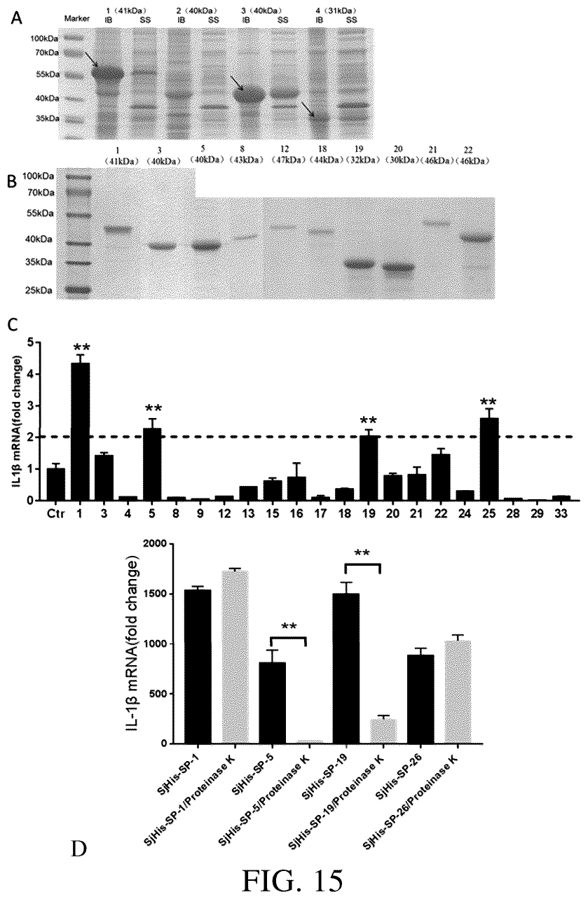

The inventors used Millipore ultrafiltration centrifuge tubes to prepare fractions with different molecular weights (f1 to f5) in the egg culture supernatant, i.e. f1<3 kDa; 3 kDa<f2<10 kDa; 10 kDa<f3<30 kDa; 30 kDa<f4<50 kDa and f5>50 kDa, and the activation effect of each fraction on macrophages was detected in vitro. The results showed that only the f4 fraction (30-50 kDa) had a significant activation effect on MH-S, and the level of Il1b mRNA expressed in MH-S activated by the f4 fraction was comparable to that activated by FES (FIG. 14 Panel A), while other fractions showed no obvious activation effect on MH-S. In vivo experiments showed that mice injected with the f4 fraction significantly inhibited lung and liver metastatic tumors, with inhibition rates of 80.8% and 94.3%, respectively (FIG. 14 Panel B, Panel C, Panel D).

### (3) Screening and Identification of Schistosome-Derived Effector Proteins for Activating MH-S

### 1) Screening Based on the Above f4 Fraction

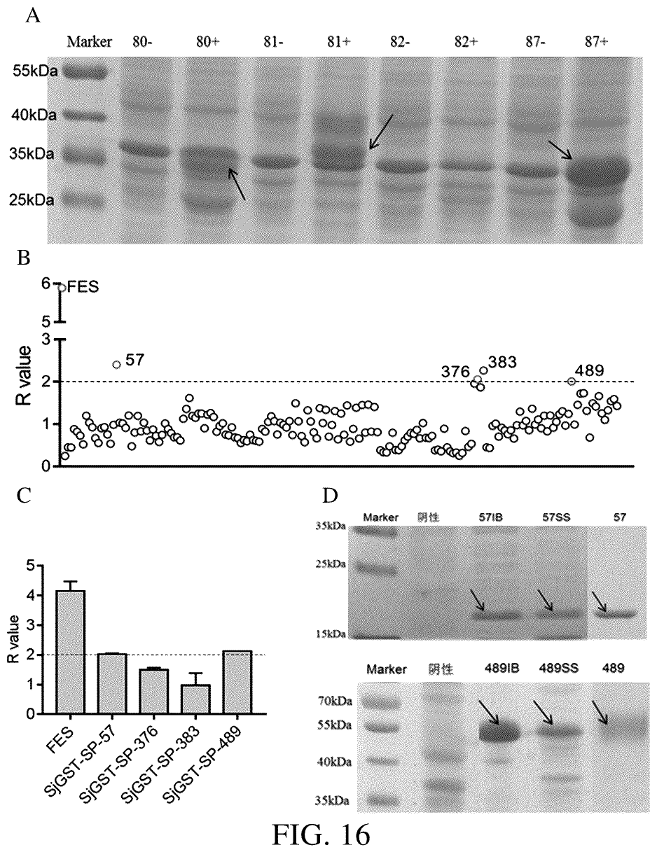

The above results indicate that the f4 fraction (30-50 kDa) is the effective component for activating MH-S. Accordingly, the inventors conducted mass spectrometry analysis on the f4 fraction. By analyzing two samples, 29 Schistosoma japonicum secreted and excreted proteins (SjSP proteins) were selected based on criteria such as matching scores and molecular weight, and recombinant expression plasmids with His tags were constructed and expressed in Escherichia coli. Among them, 21 SjSP recombinant proteins were successfully expressed (FIG. 15 Panel A) and subsequently purified using a nickel column (FIG. 15 Panel B). Each recombinant protein was cultured with MH-S and the expression level of IL-1β in MH-S was detected. The preliminary screening results showed that four SjSP proteins had the ability to stimulate the upregulation of IL-1β expression (FIG. 15 Panel C). Further screening and identification, including the use of protease digestion treatment, revealed that the ability of two proteins (SjHisSP-5 and SjHisSP-19) to stimulate IL-1β expression in MH-S was sensitive to protease digestion (FIG. 15 Panel D). Thus, SjHisSP-5 and SjHisSP-19 proteins were selected for subsequent animal experiments.

### 2) Screening Based on the Pre-Established SjSP Protein Library in the Laboratory

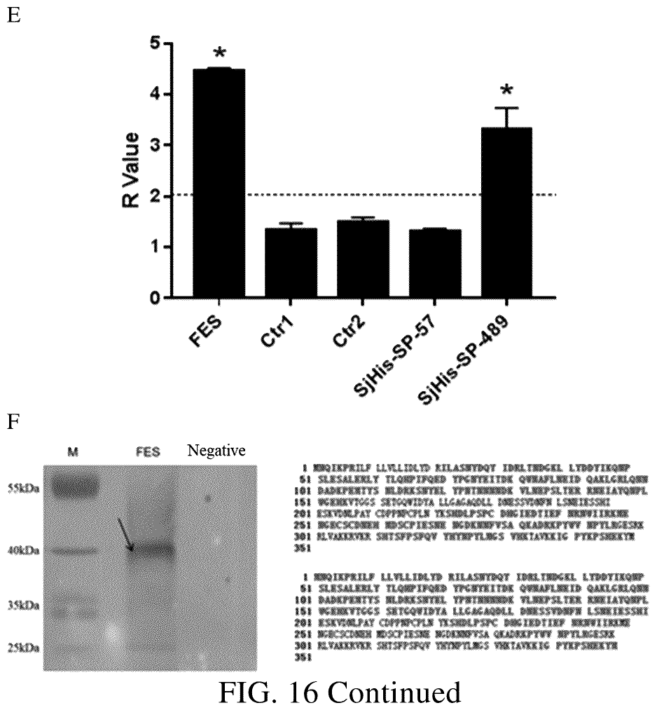

Previously, a screening library consisting of 205 secreted and excreted proteins of Schistosoma japonicum has been constructed by the laboratory. According to recent literature reports, the inventors further added 27 secreted and excreted proteins of eggs or Schistosoma japonicum circulating antigens. These 232 secreted and excreted proteins were subjected to fusion expression with GST (Glutathione S-transferase), and successfully expressed in the E. coli system (FIG. 16 Panel A). Among these, 183 SjGST-SP fusion proteins were co-cultured with MH-S cells, and the level of IL-1β in the culture supernatant was detected using sandwich ELISA. The preliminary screening results showed that four SjGST-SP could upregulate the expression and secretion of IL-1β in MH-S (R≥2.0) (FIG. 16 Panel B). Further repeated screening and verification confirmed that two of these proteins (SjGST-SP-489 and SjGST-SP-57) had the ability to activate MH-S and upregulate its IL-1β secretion (FIG. 16 Panel C). On this basis, GST-deleted recombinant proteins SjHis-SP-57 and SjHis-SP-489 were reconstructed (FIG. 16 Panel D). The results of in vitro MH-S activation experiments showed that the GST-deleted SjHis-SP-489 still retained the ability to activate MH-S and was sensitive to protease digestion (FIG. 16 Panel D), while SjHis-SP-57 lost its ability to upregulate the expression and secretion of IL-1β (FIG. 16 Panel E). The amino acid sequence of the purified SjSP-489 recombinant protein was analyzed by mass spectrometry, and the results of mass spectrometry analysis of two samples covered 94.67% of the amino acid sequence of the SjSP-489 recombinant protein (FIG. 16 Panel F), further verifying the sequence of the SjSP-489 recombinant protein.

### 3) Inhibition of Mouse Metastatic Tumors by SjHis-SP-19 and SjHis-SP-489

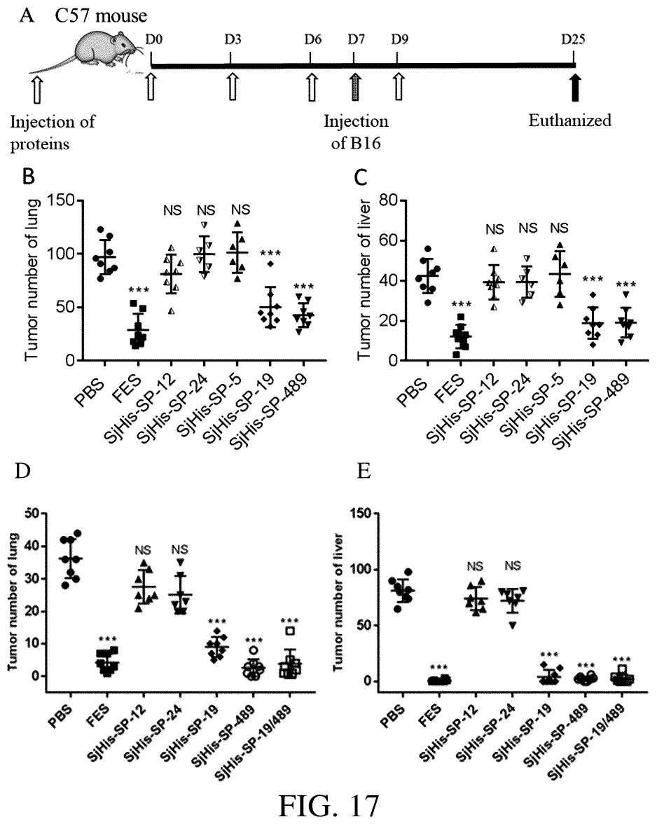

The above in vitro screening experiments have identified three recombinant proteins (SjHis-SP-5, SjHis-SP-19, and SjHis-SP-489). To further confirm whether these recombinant proteins could produce anti-tumor effects in vivo, the inventors used the above-mentioned mouse tumor model to conduct experimental observations on the in vivo immune effects of these three proteins. FES was used as the positive control, and two additional recombinant proteins (SjHis-SP-12 and SjHis-SP-24) that tested negative in the in vitro screening served as negative controls. Following the experimental procedure and time points in FIG. 17 Panel A, each mouse was inoculated with a dose of 30 μg of each recombinant protein, delivered via the tail vein in 4 separate injections. After 16 days, the number of tumors in the mouse lungs and liver was observed. The results showed that the average numbers of tumors in the lungs and liver of the PBS group were 97.1±16.0 and 42.3±8.5, respectively; Compared with the PBS group, the FES positive control significantly inhibited tumor growth in the lungs and liver, with average tumor numbers of 28.9±15.1 and 12.1±5.9, respectively, representing a reduction of 70.3% and 71.4% in tumor number, respectively (P<0.01) (FIG. 17 Panel B, Panel C). Among the three proteins that tested positive in the in vitro screening, SjHis-SP-19 and SjHis-SP-489 proteins significantly inhibited tumor growth in mice, with average tumor numbers of 50.2±18.8 and 42.7±11.2 in the lungs (P<0.01), representing a reduction of 48.2% and 51.0%, respectively (P<0.01); and with average tumor numbers of 18.9±7.9 and 19.7±7.4 in the liver, representing a reduction of 55.4% and 54.9%, respectively (P<0.01). Compared with the control group, they could significantly inhibit tumor growth in the lungs and liver (P<0.01) (FIG. 17 Panel B, Panel C); However, the SjSP-5 protein had no significant inhibitory effect on liver and lung tumors in mice. The negative controls SjHis-SP-12 and SjHis-SP-24 also showed no inhibitory effect (FIG. 17 Panel B, Panel C).

Next, the inventors conducted a combined immunization of SjHis-SP-19 and SjHis-SP-489 proteins. The total dose of the two combined proteins was 60 μg per mouse per administration, and the dose of each individual protein in the SjHis-SP-19 and SjHis-SP-489 single protein groups was also increased to 60 μg per mouse per administration. The results showed that the average numbers of tumors in the lungs and liver of the PBS group were 36.2±6.0 and 81.2±10.1, respectively (FIG. 17 Panel D, Panel E). Compared with the PBS group, the FES positive control significantly inhibited the growth of tumors in the lungs and liver, with average tumor numbers of 4.2±2.7 and 0.6±1.0, respectively (P<0.01), representing a reduction of 88.3% and 99.2%, respectively; 60 μg of SjHis-SP-19 and SjHis-SP-489 proteins significantly inhibited tumor growth in mice, with average numbers of tumors in the lungs of 9.0±3.0 and 4.1±6.2, respectively (P<0.01), representing a reduction of 75.2% and 95.0%, respectively (FIG. 17 Panel D), and with average numbers of tumors in the liver of 4.1±6.2 and 2.4±2.0, respectively (P<0.01) (FIG. 17 Panel E), representing a reduction of 94.9% and 97.0%, respectively. Their inhibitory efficacy was comparable to that of the FES positive control (FIG. 17 Panel D, Panel E). These experiments further verified the in vivo anti-tumor effects of SjHis-SP-19 and SjHis-SP-489 proteins in mice. The combination of 30 μg SjHis-SP-19 and 30 μg SjHis-SP-489 could produce potent anti-tumor effects, while the negative controls of 60 μg SjHis-SP-12 and SjHis-SP-24 did not show any inhibitory effect on tumor growth (FIG. 17 Panel D, Panel E).

### 4) the Role of Recombinant Effector Proteins in Activating Alveolar Macrophages In Vitro and In Vivo

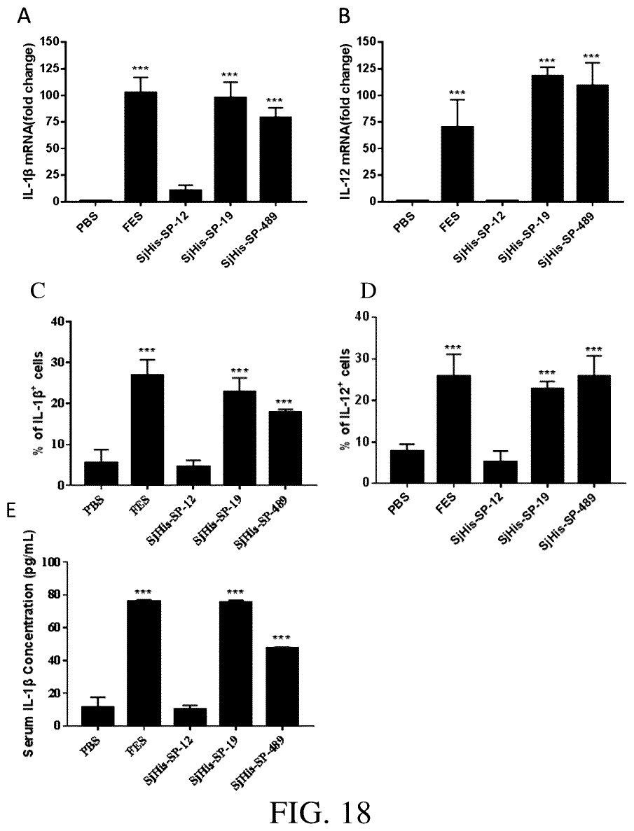

The above research indicates that SjHis-SP-19 and SjHis-SP-489 proteins can induce anti-tumor effects in vivo similar to those of FES, but does this effect occur through the activation of mouse alveolar macrophages? In this regard, FES was used as the positive control and SjHis-SP-12 protein was used as the negative control. A final concentration of 20 μg/ml each protein was added to the culture medium of mouse alveolar macrophages, and the mRNA expression levels of Il1b and Il12b in the cells were detected after 24 hours of cultivation. The results showed that compared with the PBS group, the mRNA expression of Il1b and Il12b in cells from the FES group was significantly increased; There was no significant difference in the mRNA expression of Il1b and Il12b in cells between the SjHis-SP-12 negative control protein group and the PBS group, while the mRNA expression of Il1b and Il12b was significantly increased in the SjHis-SP-19 and SjHis-SP-489 protein groups (P<0.01, FIG. 18 Panel A, Panel B).

In order to further demonstrate that SjHis-SP-19 and SjHis-SP-489 proteins can activate alveolar macrophages in mice, the inventors injected proteins at a dose of 60 μg/mouse through the tail vein into the mice for a total of 4 injections (D0, D3, D6, and D9), and then observed the functional changes of alveolar macrophages in the mice. The results showed that FES significantly upregulated the proportion of alveolar macrophages expressing IL-1β and IL-12 (FIG. 18 Panel C, Panel D), while increasing the serum IL-1β level in mice (FIG. 18 Panel E). The negative protein control group had no such effect (FIG. 18 Panel C, Panel D, Panel E). These results indicate that the recombinant proteins SjHis-SP-19 and SjHis-SP-489 can activate mouse M1-type alveolar macrophages in vivo, suggesting that these two proteins exert in vivo anti-tumor effects by activating M1-type alveolar macrophages.

### Example 9: Homology Analysis of SjHis-SP-19 and SjHis-SP-489 Sequences

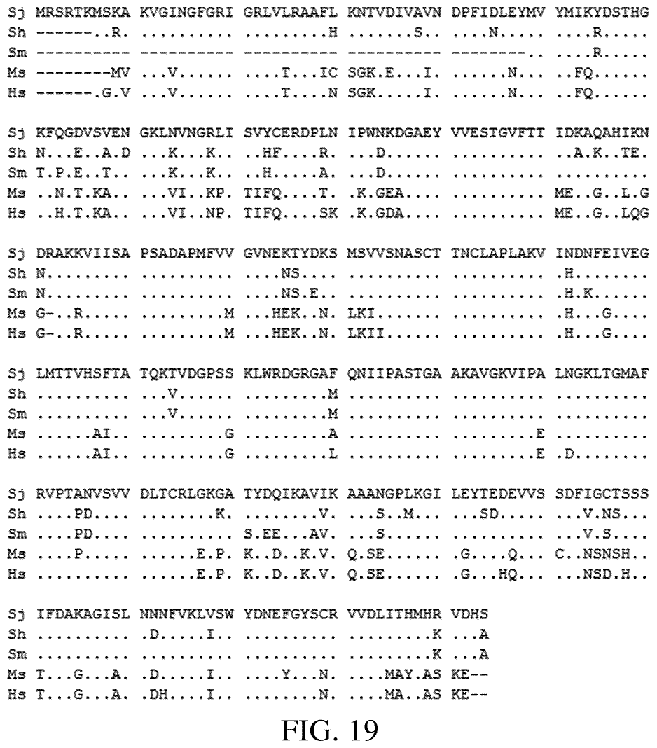

SjSP-19 (FN316857) encodes glyceraldehyde 3-phosphate dehydrogenase (GAPDH). The inventors analyzed the homology of Schistosoma japonicum GAPDH with that of Schistosoma haematobium and Schistosoma mansoni. Among schistosome species, the amino acid sequence similarity of this protein is 85.5%. Compared with GAPDH in humans and mice, the amino acid sequence similarity of this protein is 70.1% (FIG. 19). The gene of this protein is a house-keeping gene, which is conserved in biological evolution.

The function of SjSP-489 (AY814009) gene in schistosomes remains unknown; however, it contains a 7B2 domain encoding a neuroendocrine protein.

In mammals, the neuroendocrine protein gene encodes a secreted chaperone protein that prevents the aggregation of other secreted proteins, including proteins associated with neurodegenerative and metabolic diseases.

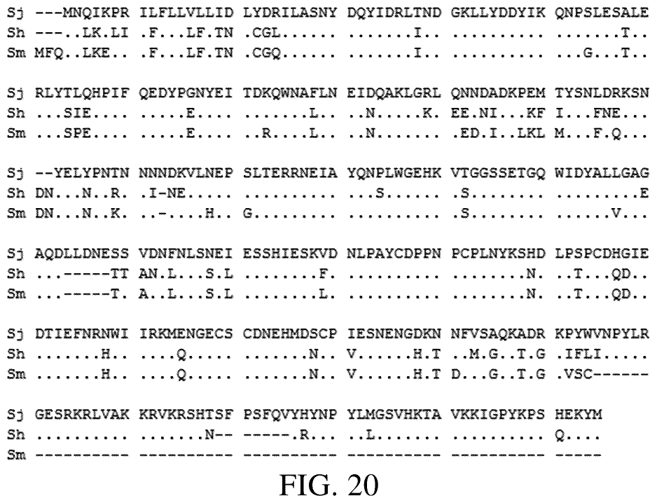

Sequence alignment shows that the sequence similarity of the SjSP-489 (AY814009) gene in schistosomes is 76.7% (FIG. 20).

The amino acid sequences of the neuroendocrine protein 7B2 in humans and mice (Human: NP_001138229.1; Mouse: NP_033188.3) show low similarity to that of SjSP-489.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. (canceled)

2. A pharmaceutical composition, comprising (a) a pharmaceutically acceptable carrier and (b) an active ingredient, wherein the active ingredient is selected from the group consisting of:
(Z1) a schistosome egg polypeptide or a coding sequence thereof, or an expression vector expressing the egg polypeptide, wherein the egg polypeptide comprises: Sj-SP-19, Sj-SP-489, or a combination thereof;
(Z2) a fraction f4 of the live fertile egg culture supernatant (FES) of schistosome, wherein the fraction f4 is prepared using Millipore ultrafiltration centrifuge tubes with molecular weight cut-offs of 30 Kd and 50 Kd, and it is essentially composed of polypeptides with a molecular weight of approximately 30-50 Kd;
(Z3) a protein fraction from the live fertile egg culture supernatant (FES) of schistosome, wherein the protein fraction is free of or substantially free of components derived from schistosome, with the exception of proteins, and is free of or substantially free of components derived from any species, with the exception of Schistosoma;
(Z4) a live fertile egg culture supernatant (FES) of schistosome;
(Z5) any combination of Z1 to Z4 mentioned above.

3. (canceled)

4. A schistosome egg polypeptide combination, which is essentially composed of Sj-SP-19 and Sj-SP-489, or a fusion protein of Sj-SP-19 and Sj-SP-489; wherein the amino acid sequence of the schistosome egg polypeptide Sj-SP-19 is as set forth in SEQ ID NO: 1, the amino acid sequence of the schistosome egg polypeptide Sj-SP-489 is as set forth in SEQ ID NO: 2.

5-6. (canceled)

7. A method for in vitro activation of alveolar macrophages, wherein alveolar macrophages are cultured in the presence of a substance to obtain activated alveolar macrophages, and the substance is selected from the group consisting of:
(Z1) a schistosome egg polypeptide or a coding sequence thereof, or an expression vector expressing the egg polypeptide, wherein the egg polypeptide comprises: Sj-SP-19 or an active ingredient thereof, Sj-SP-489 or an active ingredient thereof, or a combination thereof;
(Z2) a fraction f4 of the live fertile egg culture supernatant (FES) of schistosome, wherein the fraction f4 is prepared using Millipore ultrafiltration centrifuge tubes with molecular weight cut-offs of 30 Kd and 50 Kd, and it is essentially composed of polypeptides with a molecular weight of approximately 30-50 Kd;
(Z3) a protein fraction from the live fertile egg culture supernatant (FES) of schistosome, wherein the protein fraction is free of or substantially free of components derived from schistosome, with the exception of proteins, and is free of or substantially free of components derived from any species, with the exception of Schistosoma;
(Z4) a live fertile egg culture supernatant (FES) of schistosome;
(Z5) any combination of Z1 to Z4 mentioned above.

8. An activated alveolar macrophage prepared using the method of claim 7.

9. A cell formulation or a pharmaceutical composition, which comprises the activated alveolar macrophage according to claim 8 and a pharmaceutically acceptable carrier.

10. A method for prevention and/or treatment of tumors, comprising a step of administrating the activated alveolar macrophage according to claim 7 to a subject in need thereof.

11. A method for (a) prevention and/or treatment of tumors; (b) activation of alveolar macrophages (AM); and/or (c) activation of innate immunity, comprising a step of administrating the pharmaceutical composition according to claim 2 to a subject in need thereof.

12. The pharmaceutical composition according to claim 2, wherein the amino acid sequence of the schistosome egg polypeptide Sj-SP-19 is as set forth in SEQ ID NO: 1.

13. The pharmaceutical composition according to claim 2, wherein the amino acid sequence of the schistosome egg polypeptide Sj-SP-489 is as set forth in SEQ ID NO: 2.


SEQUENCE LISTING (6 sequences)

SEQ ID NO: 1 - protein, 296 aa, Schistosoma japonicum
MVYMIKYDSTHGKFQGDVSVENGKLNVNGRLISVYCERDPLNIPWNKDGAEYVVESTGVFTTIDKAQAHIKNDRAKKVIISAPSADAPMFVVGVNEKTYDKSMSVVSNASCTTNCLAPLAKVINDNFEIVEGLMTTVHSFTATQKTVDGPSSKLWRDGRGAFQNIIPASTGAAKAVGKVIPALNGKLTGMAFRVPTANVSVVDLTCRLGKGATYDQIKAVIKAAANGPLKGILEYTEDEVVSSDFIGCTSSSIFDAKAGISLNNNFVKLVSWYDNEFGYSCRVVDLITHMHRVDHS

SEQ ID NO: 2 - protein, 350 aa, Schistosoma japonicum
MNQIKPRILFLLVLLIDLYDRILASNYDQYIDRLTNDGKLLYDDYIKQNPSLESALERLYTLQHPIFQEDYPGNYEITDKQWNAFLNEIDQAKLGRLQNNDADKPEMTYSNLDRKSNYELYPNTNNNNDKVLNEPSLTERRNEIAYQNPLWGEHKVTGGSSETGQWIDYALLGAGAQDLLDNESSVDNFNLSNEIESSHIESKVDNLPAYCDPPNPCPLNYKSHDLPSPCDHGIEDTIEFNRNWIIRKMENGECSCDNEHMDSCPIESNENGDKNNFVSAQKADRKPYWVNPYLRGESRKRLVAKKRVKRSHTSFPSFQVYHYNPYLMGSVHKTAVKKIGPYKPSHEKYM

SEQ ID NO: 3 - protein, 330 aa, synthetic construct
MGSSHHHHHHSSGLVPRGSHMASMTGGQQMGRGSMVYMIKYDSTHGKFQGDVSVENGKLNVNGRLISVYCERDPLNIPWNKDGAEYVVESTGVFTTIDKAQAHIKNDRAKKVIISAPSADAPMFVVGVNEKTYDKSMSVVSNASCTTNCLAPLAKVINDNFEIVEGLMTTVHSFTATQKTVDGPSSKLWRDGRGAFQNIIPASTGAAKAVGKVIPALNGKLTGMAFRVPTANVSVVDLTCRLGKGATYDQIKAVIKAAANGPLKGILEYTEDEVVSSDFIGCTSSSIFDAKAGISLNNNFVKLVSWYDNEFGYSCRVVDLITHMHRVDHS

SEQ ID NO: 4 - protein, 384 aa, synthetic construct
MGSSHHHHHHSSGLVPRGSHMASMTGGQQMGRGSMNQIKPRILFLLVLLIDLYDRILASNYDQYIDRLTNDGKLLYDDYIKQNPSLESALERLYTLQHPIFQEDYPGNYEITDKQWNAFLNEIDQAKLGRLQNNDADKPEMTYSNLDRKSNYELYPNTNNNNDKVLNEPSLTERRNEIAYQNPLWGEHKVTGGSSETGQWIDYALLGAGAQDLLDNESSVDNFNLSNEIESSHIESKVDNLPAYCDPPNPCPLNYKSHDLPSPCDHGIEDTIEFNRNWIIRKMENGECSCDNEHMDSCPIESNENGDKNNFVSAQKADRKPYWVNPYLRGESRKRLVAKKRVKRSHTSFPSFQVYHYNPYLMGSVHKTAVKKIGPYKPSHEKYM

SEQ ID NO: 5 - DNA, 890 bp, Schistosoma japonicum
  source 1..890: mol_type = genomic DNA
atggtgtacatgataaaatatgactccacccatggaaagtttcaaggtgatgtttcggttgagaacggaaaacttaatgtcaatggaaggcttatatcagtttactgcgagagggatccattgaacataccatggaacaaggatggtgctgagtatgttgtagagtccactggagtcttcactacaattgataaagctcaagctcatattaaaaacgatcgggctaaaaaagttataatatcagctccctcggcagacgcacccatgtttgttgttggtgtgaatgaaaagacttacgacaagtcaatgtctgtggtttcgaatgcatcgtgcaccacaaactgtctagcacctctagctaaagtcattaatgacaattttgaaatagttgaaggccttatgactactgtacactcatttacggctacgcaaaagaccgttgatggaccatcttcaaaactgtggagagatggtcgtggggcgtttcagaatattattccagcctccactggtgctgcaaaggcagtgggcaaagtcatccctgcattaaacggaaagttgacaggaatggctttccgggtgcctacagcgaatgtttcagtagttgacctgacatgcagattgggcaaaggagctacctacgatcaaatcaaggctgtgatcaaagcagccgcaaatggaccattaaaaggcatcttggaatatactgaagatgaagttgtcagctcagactttattggatgtaccagttcatccatatttgatgcaaaggctggaatctctctcaacaacaatttcgtgaaactggtttcatggtacgacaatgaattcggctacagttgccgcgtggtcgatctcatcacgcatatgcatagagtcgaccattctta

SEQ ID NO: 6 - DNA, 1053 bp, Schistosoma japonicum
  source 1..1053: mol_type = genomic DNA
atgaaccaaatcaaacctagaatattatttctgttagtgcttttaattgatctgtatgatcgaatattagcaagtaattatgatcagtatatagatagattgacaaatgatggcaaattattatacgatgattatattaaacagaatcctagtttagaatcagcattagaacgattatatacgctacaacatccaatttttcaagaagattatccaggaaattatgagattactgataaacaatggaatgcatttctaaatgaaatcgatcaagctaaattaggcagactgcaaaacaatgatgctgataaaccagagatgacctactcaaatctcgacagaaaatcgaattatgaattgtatccaaatacaaataataataatgacaaagttttaaatgaacctagtttaacagaacgtcgaaatgaaatcgcctatcaaaatccactgtggggtgaacataaagttactggtggttccagtgaaacaggtcaatggatagattatgctttattaggagctggagcacaagatctacttgataatgaatcatcagttgataattttaatctttccaatgaaatagaatcatctcatatagagtcaaaagttgataatttaccagcatattgtgatccacctaatccttgtccattaaattataaatcacatgatttaccgtcaccatgtgatcatggtattgaagatactatcgagtttaatcgaaactggataataaggaaaatggaaaatggtgaatgttcatgtgacaatgaacatatggatagttgcccaattgaatcaaatgaaaatggagacaaaaataattttgtttcagcacaaaaggcggatagaaaaccatactgggttaatccatatctccggggtgaaagtcgaaaaaggctcgtagctaagaaacgagtaaagcgttcacatacttcttttccatcttttcaggtataccattacaatccttatctgatgggtagcgttcataaaacagcagtgaaaaaaattggaccatacaaaccatcccatgaaaaatatatgtaa